(19) United States
(12) Patent Application Publication
(10) Pub. No.: US 2025/0250581 A1
(43) Pub. Date: Aug. 7, 2025

(54) HERBICIDE-RESISTANT POLYPEPTIDE AND APPLICATION THEREOF

(71) Applicant: SHANDONG SHUNFENG BIOTECHNOLOGY CO.LTD., Jinan (CN)

(72) Inventors: Mugui WANG, Jinan (CN); Jinshan ZHANG, Jinan (CN)

(73) Assignee: SHANDONG SHUNFENG BIOTECHNOLOGY CO.LTD., Jinan (CN)

(21) Appl. No.: 18/856,632

(22) PCT Filed: Apr. 11, 2023
(86) PCT No.: PCT/CN2023/087546

§ 371 Date: Oct. 14, 2024

(30) Foreign Application Priority Data
Apr. 14, 2022 (CN) 202210387532.X

(51) Int. Cl. C12N 15/82; C12N 9/12; C12N 9/22; C12N 15/11

(52) U.S. Cl. CPC C12N 15/8274; C12N 9/12; C12N 9/226; C12N 15/11; C12N 15/8213; C12N 2310/20

(57) ABSTRACT

Herbicide-resistant genes, polypeptides and their applications in plant breeding are provided. Specifically, the disclosure provides an application of an HIR1 protein in the preparation of a herbicide-resistant plant. After the HIR1 protein of a plant is overexpressed, the plant has strong resistance to herbicide, and has very broad application prospects in improving and cultivating the herbicide-resistant plant.

### TECHNICAL FIELD

The invention belongs to the field of agricultural genetic engineering, and specifically relates to herbicide-resistant genes, polypeptides, and their application in plant breeding.

### BACKGROUND TECHNOLOGY

4-hydroxyphenylpyruvate dioxygenase (HPPD, EC 1.13.11.27) is an important enzyme in the process of tyrosine metabolism in organisms and exists in almost all aerobic organisms. Tyrosine in the organism generates p-hydroxyphenylpyruvic acid (HPPA) under the action of tyrosine aminotransferase (TAT). HPPD can catalyze the conversion of HPPA into homogentisate (HGA) with the participation of oxygen. In animals, the main function of HPPD is to promote the catabolism of tyrosine, arylamine, and phenylalanine. However, the role in plants is significantly different from that in animals. The HGA further forms plastoquinones and tocopherols (vitamin E). Tocopherol acts as a related antioxidant and is essential for plant growth, which can effectively enhance the stress resistance of plants. Plastoquinone is a key cofactor in the photosynthesis process of plants, promoting the synthesis of carotenoids and other substances in plants. More than 60% of chlorophyll in plants is bound to light-harvesting antenna complexes, which absorb sunlight energy and transfer excitation energy to the photosynthesis reaction center. Carotenoids are an important component of the chlorophyll-binding protein and antenna system at the reaction center, serving as light-absorbing accessory pigments in plant photosynthesis, having the ability to absorb and transfer electrons, and playing an important role in scavenging free radicals.

HPPD inhibition will lead to photosynthesis uncoupling and a deficiency of accessory light-harvesting pigments in plant cells. At the same time, due to the lack of photoprotection usually provided by carotenoids, reactive oxygen intermediates and photooxidation lead to chlorophyll destruction, resulting in bleaching symptoms in plant photosynthetic tissues, growth inhibition, and even death.

Identified as a herbicide target since the 1990s, HPPD is the successor to acetolactate synthase (ALS), 5-enolpyruvylshikimate-3-phosphate synthase (EPSPS), and acetyl-CoA carboxylase (ACCase). Its unique mechanism of action can effectively control a variety of resistant weeds. HPPD herbicides are a hot-selling product that has emerged in recent years and have a series of advantages, such as high efficiency, low toxicity, good environmental compatibility, and high safety for after reap crops. Studies have found that there are significant differences in the homology of HPPD amino acid sequences between plants and mammals, while the homology of the same plant kingdom or the same animal kingdom is relatively high. This provides a theoretical basis for the subsequent development of HPPD herbicides with higher selectivity and safety. At present, five herbicides targeting HPPD have been developed according to structural classification, mainly including triketones, pyrazolones, isoxazoles, diketonitriles, and benzophenones.

However, these HPPD-inhibiting herbicides may cause some damage to crops while killing weeds. Different crops have different tolerances to different HPPD herbicides, which also limits the scope of use of HPPD herbicides. Therefore, it is particularly important to obtain crops that tolerate herbicides. In addition to trying to prevent the production of HPPD-mediated homogentisate, current strategies also include overexpressing the enzyme to produce a large number of herbicide target enzymes in plants to reduce the inhibitory effect of herbicides. Although overexpression of HPPD makes plants more tolerant to herbicides (such as diketonitrile derivatives of isoxaflutole) before germination, this tolerance is not enough to resist post-germination herbicide treatment.

Clustered regularly interspaced short palindromic repeats (CRISPR)/CRISPR-associated protein (Cas) gene editing technology has emerged as a genetic engineering technology in recent years. It is a DNA cutting technology mediated by guide RNA. A variety of editing systems have been developed for different Cas, including Cas9, Cpf1, Cms1, C2c1, C2c2, etc. CRISPR/Cas editing technology can achieve three types of site-directed editing: the first is site-directed knockout of genes. Cas proteins recognize and cut targets under the guidance of guide RNA (gRNA), resulting in double-stranded DNA breaks; broken DNA is usually repaired by non-homologous end joining (NHEJ); frameshift mutations are easily generated during repair to destroy the gene. The efficiency of site-directed knockout is relatively high. The second is to replace the target sequence or perform site-directed insertion by homologous replacement of the target. When double-stranded DNA breaks occur, if there is a homologous repair template nearby, homologous replacement or site-directed insertion may occur. The efficiency of homologous replacement is low and becomes lower as the length of the sequence to be replaced increases. The third is single-base editing. Single-base editing is a gene editing method that uses the CRISPR/Cas system to target deaminase at a specific site in the genome to modify a specific base. This method has been successfully used in rice.

Due to the short period of large-scale use of HPPD herbicides, there are currently very few reports on HPPD resistance genes. In 2019, Japanese scientists discovered a rice gene HIS1 (HPPD INHIBITOR SENSITIVE 1) that makes rice resistant to HPPD herbicides, but this gene is only resistant to triketone herbicides and has no effect on other types of HPPD herbicides. Therefore, there is an urgent need to explore and utilize the potential resistance gene resources of crops to enhance tolerance to HPPD inhibitors.

### CONTENT OF THE INVENTION

The present invention provides an application of HIR1 protein in preparing a herbicide-resistant plant, and the plant has strong resistance to herbicides after the HIR1 gene protein is overexpressed.

In one aspect, the present invention provides an application of HIR1 protein in preparing a herbicide-resistant plant or in conferring or enhancing plant resistance to herbicides, and the protein includes any one of the following groups or a combination thereof:
i. the HIR1 protein is a homologous protein of the protein shown in SEQ ID No.1;
ii. the amino acid sequence of the HIR1 protein has at least 50% sequence identity compared to the sequence shown in any one of SEQ ID No. 1-7; and
iii. the HIR1 protein includes the amino acid sequence shown in any one of SEQ ID No.1-7.

In a preferred example, the amino acid sequence of the HIR1 protein has at least 50%, at least 60%, at least 70%, at least 75%, at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity compared to the sequence shown in any one of SEQ ID No.1-7, and has substantially the same function as the protein shown in any one of SEQ ID No.1-7.

In one embodiment, the HIR1 protein is derived from a monocot or a dicot, for example, rice, corn, sorghum, Panicum miliaceum, wheat, or barley.

In the present invention, the HIR1 protein includes a protein encoded by the gene LOC_Os02g09720 from rice, annotated as a multidrug resistance protein of unknown function, and the amino acid sequence is shown in SEQ ID No.1.

In one embodiment, the protein homologous to the above HIR1 protein also includes a protein from rice (NCBI Reference Sequence: XP_015625026.1), annotated as a putative multidrug resistance protein, and the amino acid sequence is shown in SEQ ID No.2, and its sequence identity with SEQ ID No. 1 is 92%.

In one embodiment, the protein homologous to the above HIR1 protein also includes a protein from Panicum miliaceum (GenBank: RLN07167.1), annotated as a putative multidrug resistance protein, and the amino acid sequence is shown in SEQ ID No.3, and its sequence identity with SEQ ID No. 1 is 91%.

In one embodiment, the protein homologous to the above HIR1 protein also includes a protein from corn (NCBI Sequence ID: AQK60189.1), annotated as ABC transporter B family member 15, and the amino acid sequence is shown in SEQ ID No.4, and its sequence identity with SEQ ID No. 1 is 91%.

In one embodiment, the protein homologous to the above HIR1 protein also includes a protein from sorghum (NCBI Reference Sequence: XP_002453447.2), annotated as a putative multidrug resistance protein, and the amino acid sequence is shown in SEQ ID No.5, and its sequence identity with SEQ ID No. 1 is 91%.

In one embodiment, the protein homologous to the above HIR1 protein also includes a protein from wheat (GenBank: KAF7078743.1), annotated as hypothetical protein CFC21_083126, and the amino acid sequence is shown in SEQ ID No.6, and its sequence identity with SEQ ID No. 1 is 90%.

In one embodiment, the protein homologous to the above HIR1 protein also includes a protein from barley (GenBank: KAE8801832.1), annotated as a putative multidrug resistance protein, and the amino acid sequence is shown in SEQ ID No.7, and its sequence identity with SEQ ID No.1 is 90%.

In one embodiment, the homologous protein of the protein shown in SEQ ID No.1 is selected from one or any combination of SEQ ID No.2-7.

In one embodiment, the HIR1 protein is derived from rice, Panicum miliaceum, corn, sorghum, wheat, or barley, and the amino acid sequence of the HIR1 protein has at least 50%, at least 60%, at least 70%, at least 75%, at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity compared to the sequence shown in any one of SEQ ID No. 1-7.

In another preferred embodiment, the amino acid sequence of the HIR1 protein includes the sequence shown in any one of SEQ ID No. 1-7.

In another preferred embodiment, the amino acid sequence of the HIR1 protein is the same or basically the same as the sequence shown in any one of SEQ ID No. 1-7.

In another preferred embodiment, the basically same is that at most 50 (preferably 1-20, more preferably 1-10, more preferably 1-5) amino acids are different, where the difference includes substitution, deletion, or addition of amino acids, and overexpression of the protein can make the plant resistant to herbicides (HPPD inhibitor herbicides).

In the present invention, the HIR1 protein is overexpressed in the plant to confer or enhance the plant's resistance to herbicides.

In the present invention, the HIR1 protein is overexpressed in the plant to prepare a herbicide-resistant plant or to confer or enhance the plant's resistance to herbicides.

Overexpression in the present invention includes increasing the expression of a gene by introducing additional copies of the target gene to increase the number of copies of the target gene in a cell, or increasing the expression of the target gene by modifying or replacing the promoter of the target gene.

In one embodiment, the “introduction” includes constructing the coding gene of the target protein into an expression vector and transferring the expression vector into the plant to express the target gene. In other embodiments, the “introduction” includes inserting the target gene into the genome of the plant; preferably, the insertion can be carried out by homologous recombination double exchange method; in one embodiment, the target gene and homologous arms can be inserted into the vector, and then the vector can be transferred into the plant, and the homologous arms can be used to undergo homologous recombination double exchange with the plant genome to insert the target gene into a suitable genomic position; in other embodiments, gene editing can also be used, for example, using the CRISPR/Cas system to cut at the desired genomic site, and inserting the target gene as an exogenous donor into the cutting site.

In one embodiment, modifying or replacing the promoter of the target gene to increase the expression of the target gene includes replacing the promoter of the target gene with a strong promoter to increase the expression of the target gene, or modifying the promoter of the target gene to increase the expression of the target gene; for example, inserting a promoter (such as a 35S promoter) into the promoter region of the target gene by gene editing. In one embodiment, the overexpression of HIR1 protein refers to an increase in the expression of HIR1 protein or an increase in the expression of the HIR1 gene.

In one embodiment, the overexpression of HIR1 protein refers to an increase in the expression of the HIR1 gene by at least 1 times, preferably at least 2 times, preferably at least 3 times, preferably at least 4 times, preferably at least 5 times, preferably at least 6 times, preferably at least 10 times, preferably at least 20 times, preferably at least 30 times, preferably at least 50 times, preferably at least 70 times, preferably at least 100 times, compared with the control.

In one embodiment, the overexpression of HIR1 protein is achieved by inserting an exogenous fragment into the promoter region of the HIR1 gene, and the exogenous fragment includes a promoter, such as a 35S promoter, a 2×35S promoter, Ubi, UBQ, SPL, EFla, RPS5A, tissue-specific promoters YAO, CDC45, rbcS, or a combination thereof.

In one embodiment, the plant overexpressing HIR1 protein is homozygous or heterozygous.

In another preferred embodiment, the plant includes crops, forestry plants, vegetables, fruits, flowers, and forage grasses (including lawn grass).

In another preferred embodiment, the plant is a monocot and/or a dicot.

In another preferred embodiment, the plant is selected from one or more plants of the following group: Gramineae, Leguminosae, Cruciferae, Solanaceae, Cucurbitaceae, Chenopodiaceae, Polygonaceae, Pedaliaceae, Asteraceae, Malvaceae, Rosaceae, Pedaliaceae, Convolvulaceae, Dioscoreaceae, Umbelliferae, Liliaceae, Zingiberaceae, and Palmaceae plants.

In another preferred embodiment, the plant is selected from one or more of the following groups: rice, soybean, Arabidopsis, tobacco, tomato, potato, corn, cotton, alfalfa, sorghum, barley, wheat, millet, sweet potato, quinoa, lettuce, rapeseed, cabbage, spinach, beet, peanut, watermelon, cabbage, strawberry, cucumber, coconut, or a combination thereof.

In another preferred embodiment, the plant is selected from one or more of the following groups: rice, soybean, Arabidopsis, corn, cotton, sorghum, barley, wheat, millet, quinoa, Panicum miliaceum, or a combination thereof.

In another preferred embodiment, the plant is rice, corn, sorghum, barley, wheat, quinoa, Arabidopsis, soybean, Panicum miliaceum, or a combination thereof.

In another preferred embodiment, the herbicide is an HPPD-inhibitor herbicide (also referred to as an HPPD inhibitory herbicide). HPPD inhibitor herbicides mainly include triketones, pyrazolones, isoxazolones, diketonitriles, and benzophenones. The triketone herbicide is preferably one or any combination of mesotrione, tembotrione, tripyrasulfone, tefuryltrione, benzobicyclon, mesotrion, sulcotrione, bicyclopyrone, quinotrione, or quinotrione-methyl; the pyrazolone herbicide is preferably one or any combination of topramezone, pyrasulfotole, pyrazoxyfen, benzofenap, pyrazolate, pyrasulfotole, or tolpyralate; the isoxazolone herbicide is preferably one or any combination of isoxaflutole, isoxachlortole, and clomazone.

In another preferred embodiment, the HPPD inhibitory herbicide is preferably one or any combination of mesotrione, isoxaflutole, tembotrione, quinotrione-methyl, topramezone, or pyrasulfotole.

In another preferred embodiment, after the HIR1 protein is overexpressed, the maximum tolerance concentration of the plant to the herbicide is increased by at least 1.5 times, preferably at least 2 times, preferably at least 3 times, preferably at least 4 times, preferably at least 5 times, preferably at least 6 times, preferably at least 10 times, preferably at least 20 times, preferably at least 30 times, preferably at least 50 times, preferably at least 100 times, preferably at least 200 times, compared with the parent plant.

In another preferred embodiment, the maximum tolerance concentration of the plant containing the overexpressed HIR1 protein to the herbicide is increased by at least 2 times, preferably 3 times, preferably 4 times, preferably 5 times, preferably 6 times, preferably 7 times, preferably 8 times, preferably 10 times, preferably 12 times, preferably 14 times, preferably 16 times, preferably 20 times, preferably 30 times, preferably 50 times, preferably 100 times, preferably 200 times, compared with the parent plant.

In another aspect, the present invention provides an isolated nucleic acid molecule encoding the HIR1 protein.

In another preferred embodiment, the nucleic acid molecule is selected from the following group: genomic sequence, cDNA sequence, RNA sequence, or a combination thereof.

In another preferred embodiment, the nucleic acid molecule is preferably single-stranded or double-stranded.

In another preferred embodiment, the nucleic acid molecule further includes an operably linked promoter.

In another preferred embodiment, the promoter is selected from the following group: a constitutive promoter, a tissue-specific promoter, an inducible promoter, or a strong promoter.

In another aspect, the present invention provides a vector including the nucleic acid molecule.

In another preferred embodiment, the vector includes a cloning vector, an expression vector, a shuttle vector, or an integration vector.

The vector may be a plasmid, a virus, a cosmid, a bacteriophage, etc., which are well known to those skilled in the art.

In another aspect, the present invention provides a host cell including the nucleic acid molecule, or the vector.

In another aspect, the present invention provides the use of the nucleic acid molecule, the vector, or the host cell in preparing a herbicide-resistant plant, or in conferring or enhancing plant resistance to herbicides.

In another aspect, the present invention provides a gene editing reagent or a transgenic reagent, which can edit a plant to overexpress the HIR1 protein.

The present invention also provides the use of the gene editing reagent, or the transgenic reagent in preparing a herbicide-resistant plant, or in conferring or enhancing plant resistance to herbicides.

In another aspect, the present invention provides a plant cell, plant tissue, plant part, or plant, where the plant cell, plant tissue, plant part, or plant includes the overexpressed HIR1 protein, or the nucleic acid molecule, or the vector, or the host cell.

In another aspect, the present invention provides a method for conferring or enhancing plant resistance to herbicides, or a method for preparing a herbicide-resistant plant, the method including overexpressing the HIR 1 protein in the plant.

In another preferred embodiment, the method includes the step of overexpressing the HIR1 protein in the plant cell, plant seed, plant tissue, plant part, or plant.

In another preferred embodiment, the plant includes crops, forestry plants, vegetables, fruits, flowers, and forage grasses (including lawn grass).

In another preferred embodiment, the plant is a monocot and/or a dicot.

In another preferred embodiment, the plant is selected from one or more plants of the following group: Gramineae, Leguminosae, Cruciferae, Solanaceae, Cucurbitaceae, Chenopodiaceae, Polygonaceae, Pedaliaceae, Asteraceae, Malvaceae, Rosaceae, Pedaliaceae, Convolvulaceae, Dioscoreaceae, Umbelliferae, Liliaceae, Zingiberaceae, and Palmaceae plants.

In another preferred embodiment, the plant is selected from one or more plants of the following group: rice, soybean, Arabidopsis, tobacco, tomato, potato, corn, cotton, alfalfa, sorghum, barley, wheat, millet, sweet potato, quinoa, lettuce, rapeseed, cabbage, spinach, beet, peanut, watermelon, cabbage, strawberry, cucumber, coconut, Panicum miliaceum, or a combination thereof.

In another preferred embodiment, the plant is selected from one or more plants of the following group: rice, soybean, Arabidopsis, corn, cotton, sorghum, barley, wheat, millet, quinoa, Arabidopsis, soybean, Panicum miliaceum, or a combination thereof.

In another preferred embodiment, the plant is rice.

In another preferred embodiment, the herbicide is an HPPD-inhibiting herbicide (also referred to as an HPPD inhibitory herbicide). HPPD-inhibiting herbicide mainly includes triketones, pyrazolones, isoxazolones, diketonitriles, and benzophenones. The triketone herbicide is preferably one or any combination of mesotrione, tembotrione, triazole, tefuryltrione, benzobicyclon, mesotrion, sulcotrione, bicyclopyrone, quinotrione, or quinotrione-methyl; the pyrazolone herbicide is preferably one or any combination of topramezone, pyrasulfotole, pyrazoxyfen, benzofenap, pyrazolate, pyrasulfotole, or tolpyralate; the isoxazolone herbicide is preferably one or any combination of isoxaflutole, isoxachlortole, and clomazone.

In another preferred embodiment, the HPPD inhibitory herbicide is preferably one or any of mesotrione, isoxaflutole, tembotrione, quinotrione-methyl, topramezone, or pyrasulfotole.

In another preferred embodiment, the method includes the following steps:
(1) providing an Agrobacterium carrying an expression vector, the expression vector includes the above-mentioned nucleic acid molecule;
(2) contacting plant cells, plant tissues, and plant parts with the Agrobacterium in step (1), thereby overexpressing the HIR1 protein and integrating it into the chromosome of the plant cell; and
(3) selecting plant cells that overexpress the HIR1 protein.

In another preferred embodiment, the method includes the following steps:
(1) providing a gene editing reagent that can perform gene editing on plants to overexpress the HIR1 protein;
(2) contacting plant cells, plant tissues, and plant parts with the gene editing reagent in step (1), thereby overexpressing the HIR1 protein; and
(3) selecting plant cells that overexpress the HIR1 protein.

Preferably, the gene editing results in the introduction of an exogenous sequence into the promoter region of the plant HIR1 protein; preferably, the exogenous sequence is an additional promoter, such as a 35S promoter, and also such as a 2×35S promoter.

In another aspect, the present invention provides a method for controlling unwanted plants at a plant cultivation site, the method including:
(1) providing a plant prepared by the method; and
(2) cultivating the plant, and applying an effective amount of an HPPD inhibitory herbicide to the cultivation site.

In one embodiment, the unwanted plant is a weed.

On the other hand, the present invention also provides a method for controlling the growth of weeds near a plant, including:
a) providing the above-mentioned herbicide-resistant plant; and
b) applying an effective amount of herbicide to the plant and the weeds near the plant, thereby controlling the weeds near the plant.

In another preferred embodiment, the plant includes crops, forestry plants, vegetables, fruits, flowers, and forage grasses (including lawn grass).

In another preferred embodiment, the plant is a monocot and/or a dicot.

In another preferred embodiment, the plant is selected from one or more plants of the following group: Gramineae, Leguminosae, Cruciferae, Solanaceae, Cucurbitaceae, Chenopodiaceae, Polygonaceae, Pedaliaceae, Asteraceae, Malvaceae, Rosaceae, Pedaliaceae, Convolvulaceae, Dioscoreaceae, Umbelliferae, Liliaceae, Zingiberaceae, and Palmaceae plants.

In another preferred embodiment, the plant is selected from one or more of the following groups: rice, soybean, Arabidopsis, tobacco, tomato, potato, corn, cotton, alfalfa, sorghum, barley, wheat, millet, sweet potato, quinoa, lettuce, rapeseed, cabbage, spinach, beet, peanut, watermelon, cabbage, strawberry, cucumber, coconut, Panicum miliaceum, or a combination thereof.

In another preferred embodiment, the plant is selected from one or more of the following groups: rice, soybean, Arabidopsis, corn, cotton, sorghum, barley, wheat, millet, quinoa, Arabidopsis, soybean, Panicum miliaceum, or a combination thereof.

In another preferred embodiment, the plant is rice.

On the other hand, the present invention also provides a herbicide-resistant plant obtained by the above method.

On the other hand, the present invention also provides a method for preparing a hybrid plant. The method includes hybridizing a first plant with a second plant to obtain the hybrid plant, where the first plant is a herbicide-resistant plant prepared by the method of the present invention.

### General Definition

Unless defined in this application, the scientific terms or professional terms used in the present invention have the meanings understood by those skilled in the art. When the meanings understood by those skilled in the art conflict with the meanings defined in this application, the meanings defined in this application shall prevail.

As used herein, the term “HPPD” refers to 4-Hydroxyphenylpyruvate Dioxygenase (HPPD, EC 1.13.11.27), which exists in various organisms and is a key enzyme that catalyzes the oxidation of 4-hydroxyphenylpyruvate (HPP), a degradation product of tyrosine, to generate homogentisate (HGA). An inhibition of HPPD can lead to photosynthesis uncoupling and a deficiency of accessory light-harvesting pigments in plant cells. At the same time, due to the lack of photoprotection usually provided by carotenoids, reactive oxygen intermediates and photooxidation lead to chlorophyll destruction, resulting in bleaching symptoms in plant photosynthetic tissues, growth inhibition, and even death. HPPD-inhibiting herbicides have been proven to be very effective selective herbicides with broad-spectrum herbicidal activity. They can be used both before and after germination and have the characteristics of high activity, low residue, safety for mammals, and environmental friendliness.

As used herein, the term “herbicide” refers to a substance that has herbicidal activity itself or is used in combination with other herbicides and/or additives that can change its effect, and is manifested as a preparation that inhibits plant growth or even kills plants.

As used herein, the terms “HPPD inhibitor”, “HPPD herbicide”, “HPPD inhibitory herbicide”, and “HPPD-inhibiting herbicide” are used interchangeably and refer to a substance that has herbicidal activity itself or is used in combination with other herbicides and/or additives that can change its effect, which works by inhibiting HPPD, and is manifested as a preparation that inhibits plant growth or even kills plants. Substances that can inhibit HPPD and act as herbicides are well known in the art and include many types, 1) triketones, for example, sulcotrione (CAS No.: 99105-77-8); mesotrione (CAS No.: 104206-82-8); bicyclopyrone (CAS No.: 352010-68-5); tembotrione (CAS No.: 335104-84-2); tefuryltrione (CAS No.: 473278-76-1); benzobicyclon (CAS No. 156963-66-5); quinotrione (CAS No. 1639426-14-4); quinotrione-methyl (CAS No.), 6-(2-hydroxy-6-oxidized cyclohex1-en-1); 2) diketonitriles, for example, 2-cyano-3-cyclopropyl-1-(2-methylsulfonyl-4-trifluoromethylphenyl)prop-1,3-dione (CAS No. 143701-75-1); No. 2-cyano-3-cyclopropyl-1-(2-methylsulfonyl-3,4-dichlorophenyl)prop-1,3-dione (CAS 212829-55-5); 2-cyano-1-[4-(methylsulfonyl)-2-trifluoromethylphenyl]-3-(1-methylcyclopropyl)prop-1,3-dione (CAS No.: 143659-52-3); 3) isoxazolones, for example, isoxaflutole (CAS No.: 141112-29-0); isoxachlortole (CAS No.: 141112-06-3); clomazone (CAS No.: 81777-89-1); 4) pyrazolones, for example, topramezone (CAS No.: 210631-68-8); pyrasulfotole (CAS No.: 365400-11-9); pyrazoxyfen (CAS No.: 71561-11-0); pyrazolate (CAS No.: 58011-68-0); benzofenap (CAS No.: 82692-44-2); bipyrazone (CAS No.: 1622908-18-2); tolpyralate (CAS No.: 1101132-67-5); fenpyrazone (CAS No.: 1992017-55-6); cypyrafluone (CAS No.: 1855929-45-1); tripyrasulfone (CAS No.: 1911613-97-2); 5) benzophenones; 6) others: lancotrione (CAS No.: 1486617-21-3); fenquinotrione (CAS No.: 1342891-70-6). The herbicide can be used to control unwanted plants (such as weeds) before and after germination, before and during planting, taking into account the type of crops or weeds to which it is applied.

The term “effective amount” or “effective concentration” means, respectively, an amount or concentration sufficient to kill similar parent (or wild-type) plants, plant tissues, plant cells, or host cells, or inhibit their growth, but the amount does not kill the herbicide-resistant plants, plant tissues, plant cells, and host cells of the present invention, or does not seriously inhibit their growth. Generally, the effective amount of a herbicide is the amount routinely used to kill the target weeds in agricultural production systems. Such amounts are known to those of ordinary skill in the art. The herbicides of the present invention are those that show herbicidal activity when applied directly to the plant or to the location of the plant at any growth stage or before planting or emergence. The observed effect depends on the plant species to be controlled, the growth stage of the plant, the application parameters of the dilution and the spray droplet size, the particle size of the solid component, the environmental conditions at the time of use, the specific compound used, the specific auxiliary agent and carrier used, the soil type, etc., and the amount of the chemical applied. As is known in the art, these factors and other factors can be adjusted to promote non-selective or selective herbicidal effects.

The term “parent nucleotide or polypeptide” refers to a nucleic acid molecule or polypeptide (protein) that can be found in nature, including wild-type nucleic acid molecules or proteins (polypeptides) that have not been artificially modified, and can also include nucleic acid molecules or proteins (polypeptides) that have been artificially modified but do not contain the content of the present invention. Its nucleotides can be obtained by genetic engineering technology, such as genome sequencing, polymerase chain reaction (PCR), etc., and its amino acid sequence can be derived from the nucleotide sequence. The “parent plant” is a plant containing the parent nucleotide or polypeptide. The “parent nucleotide or polypeptide” can be extracted from the parent plant according to techniques well known to those skilled in the art or can be obtained by chemical synthesis.

The “tolerance” or “resistance” described in the present invention refers to the ability of a plant to withstand herbicides under the condition of plant growth, which can generally be characterized by parameters such as the amount or concentration of the herbicide used. Furthermore, a plant that “confers herbicide resistance” or “enhanced herbicide resistance” refers to a plant whose tolerance or resistance to the herbicide is improved compared to the parent plant, and whose tolerance concentration is at least 1.5 to 200 times higher than the tolerance concentration of the parent plant. The optimal degree of improving “tolerance” or “resistance” described in the present invention is that at the same herbicide usage or concentration, it can reduce, inhibit or kill unwanted plants but does not affect the growth or viability of the plant containing the mutant protein described in the present invention.

The “conferring herbicide resistance” described in the present invention includes overexpressing the HIR1 protein in parent plants that have no resistance or tolerance to herbicides, or parent plants that have a certain or lower tolerance to herbicides (at the same herbicide concentration), thereby giving a certain degree of herbicide resistance or tolerance to plants without resistance and improving the tolerance of plants with a certain or lower tolerance to herbicides.

The terms “protein”, “protein”, “polypeptide”, and “peptide” are used interchangeably in the present invention to refer to polymers of amino acid residues, including polymers in which one or more amino acid residues are chemical analogs of natural amino acid residues. The proteins and polypeptides of the present invention can be produced by recombination or by chemical synthesis.

The terms “homology” or “identity” are used to refer to the matching of sequences between two polypeptides or between two nucleic acids. Therefore, the compositions and methods of the present invention also include homologs of the nucleotide sequences and polypeptide sequences of the present invention. “Homology” can be calculated by known methods including, but not limited to, Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M. and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

The specific amino acid positions (numbers) within the proteins of the present invention are determined by aligning the amino acid sequence of the target protein with SEQ ID NO.1 using standard sequence alignment tools, such as using the Smith-Waterman algorithm or the CLUSTALW2 algorithm to align the two sequences, where the sequences are considered aligned when the alignment score is the highest. The alignment score can be calculated according to the method described in Wilbur, W. J. and Lipman, D. J. (1983) Rapid similarity searches of nucleic acid and protein data banks. Proc. Natl. Acad. Sci. USA, 80:726-730. The default parameters are preferably used in the ClustalW2 (1.82) algorithm: protein gap opening penalty=10.0; protein gap extension penalty=0.2; protein matrix=Gonnet; protein/DNA end gap=−1; protein/DNAGAPDIST=4. The position of a specific amino acid in the protein of the present invention is preferably determined by comparing the amino acid sequence of the protein with SEQ ID No. 1 using the AlignX program (part of the vectorNTI suite) with default parameters suitable for multiple alignments (gap opening penalty: 10og gap extension penalty 0.05).

The term “encoding” refers to the inherent property of a specific nucleotide sequence in a polynucleotide, such as a gene, cDNA, or mRNA, as a template in biosynthesis with defined nucleotide sequences (i.e., rRNA, tRNA, and mRNA) or defined amino acid sequences, and in the synthesis of other polymers and macromolecules in biological processes. Therefore, if the transcription and translation of the mRNA corresponding to the gene produce a protein in a cell or other biological system, the gene encodes the protein.

The term “amino acid” refers to a carboxylic acid containing an amino group. Various proteins in organisms are composed of 20 basic amino acids.

In the present invention, amino acid residues can be represented by a single letter or by three letters, for example: alanine (Ala, A), valine (Val, V), glycine (Gly, G), leucine (Leu, L), glutamine (Gln, Q), phenylalanine (Phe, F), tryptophan (Trp, W), tyrosine (Tyr, Y), aspartic acid (Asp, D), asparagine (Asn, N), glutamic acid (Glu, E), lysine (Lys, K), methionine (Met, M), serine (Ser, S), threonine (Thr, T), cysteine (Cys, C), proline (Pro, P), isoleucine (Ile, I), histidine (His, H), arginine (Arg, R).

In the present invention, HIR1 protein refers to HPPD INHIBITOR RESISTANCE 1 protein, the gene number of the HIR1 protein in the rice genome is LOC_Os02g09720, which encodes 1,245 amino acids and is annotated as a multidrug resistance protein of unknown function. Blast analysis of the amino acid sequence of HIR1 protein shows that the gene is a protein member of the ABC transporter family, and the amino acid sequence of HIR1 protein in rice is shown in SEQ ID No.1.

In the present invention, HIR1 protein can be derived from any plant, especially monocots or dicots.

Preferably, the HIR1 protein of the present invention is derived from the genus Oryza, especially rice. More preferably, the parent HIR1 protein has the amino acid sequence shown in SEQ ID NO.1, or an amino acid sequence with at least 50%, at least 60%, at least 70%, at least 80%, at least 85%, at least 90%, at least 95%, at least 96%, at least 97%, at least 98%, or at least 99% sequence identity with the amino acid sequence shown in SEQ ID NO.1.

In the present invention, the HIR1 protein includes a protein encoded by the gene LOC_Os02g09720 from rice, annotated as a multidrug resistance protein of unknown function, and the amino acid sequence is shown in SEQ ID No. 1.

In one embodiment, the protein homologous to the above HIR1 protein also includes a protein from rice (NCBI Reference Sequence: XP_015625026.1), annotated as a putative multidrug resistance protein, and the amino acid sequence is shown in SEQ ID No.2, and its sequence identity with SEQ ID No. 1 is 92%.

In one embodiment, the protein homologous to the above HIR1 protein also includes a protein from Panicum miliaceum (GenBank: RLN07167.1), annotated as a putative multidrug resistance protein, and the amino acid sequence is shown in SEQ ID No.3, and its sequence identity with SEQ ID No. 1 is 91%.

In one embodiment, the protein homologous to the above HIR1 protein also includes a protein from corn (Sequence ID: AQK60189.1), annotated as ABC transporter B family member 15, and the amino acid sequence is shown in SEQ ID No.4, and its sequence identity with SEQ ID No. 1 is 91%.

In one embodiment, the protein homologous to the above HIR1 protein also includes a protein from sorghum (NCBI Reference Sequence: XP_002453447.2), annotated as a putative multidrug resistance protein, and the amino acid sequence is shown in SEQ ID No.5, and its sequence identity with SEQ ID No. 1 is 91%.

In one embodiment, the protein homologous to the above HIR1 protein also includes a protein from wheat (GenBank: KAF7078743.1), annotated as hypothetical protein CFC21_083126, and the amino acid sequence is shown in SEQ ID No.6, and its sequence identity with SEQ ID No. 1 is 90%.

In one embodiment, the protein homologous to the above HIR1 protein also includes a protein from barley (GenBank: KAE8801832.1), annotated as a putative multidrug resistance protein, and the amino acid sequence is shown in SEQ ID No.7, and its sequence identity with SEQ ID No.1 is 90%.

The HIR1 protein of the present invention also includes active fragments, variants, derivatives, and analogs thereof, including substances produced by any substitution, mutation, or modification of the HIR1 protein.

It is clear to those skilled in the art that the structure of a protein can be changed without adversely affecting its activity and functionality, for example, one or more conservative amino acid substitutions can be introduced into the amino acid sequence of a protein without adversely affecting the activity and/or three-dimensional structure of the protein molecule. Examples and embodiments of conservative amino acid substitutions are clear to those skilled in the art. Specifically, the amino acid residue can be substituted with another amino acid residue belonging to the same group as the amino acid residue at the site to be substituted, that is, a non-polar amino acid residue can be substituted for another non-polar amino acid residue, a polar uncharged amino acid residue can be substituted for another polar uncharged amino acid residue, a basic amino acid residue can be substituted for another basic amino acid residue, and an acidic amino acid residue can be substituted for another acidic amino acid residue. Such substituted amino acid residues may or may not be encoded by the genetic code. Conservative substitutions in which one amino acid is replaced by another amino acid belonging to the same group fall within the scope of the present invention as long as the substitution does not result in the inactivation of the biological activity of the protein. Therefore, the protein of the present invention may contain one or more conservative substitutions in the amino acid sequence, and these conservative substitutions are preferably generated by substitution according to Table 1. In addition, the present invention also encompasses proteins that also contain one or more other non-conservative substitutions, as long as the non-conservative substitutions do not significantly affect the desired function and biological activity of the protein of the present invention. Conservative amino acid substitutions can be made at one or more predicted non-essential amino acid residues. “Non-essential” amino acid residues are amino acid residues that can be changed (absence, substitution, or replacement) without changing the biological activity, while “essential” amino acid residues are required for biological activity. “Conservative amino acid substitutions” are substitutions in which an amino acid residue is replaced by an amino acid residue with a similar side chain. Amino acid substitutions can be made in non-conserved regions of a protein. Generally speaking, such substitutions are not made for conserved amino acid residues or amino acid residues located within a conserved motif, where such residues are required for protein activity. However, it should be understood by those skilled in the art that functional variants can have fewer conservative or non-conservative changes in conserved regions.

It is well known in the art that one or more amino acid residues can be changed (substituted, deleted, truncated, or inserted) from the N and/or C termini of a protein while still retaining its functional activity. Therefore, proteins in which one or more amino acid residues are changed from the N and/or C termini of a protein while retaining its desired functional activity are also within the scope of the present invention. These changes may include changes introduced by modern molecular methods such as PCR, which includes PCR amplification capable of altering or extending a protein coding sequence, with the aid of an oligonucleotide used in the PCR process, the oligonucleotide including the original amino acid coding sequence of the altered or extended protein.

It should be recognized that proteins can be altered in various ways, including amino acid substitution, deletion, truncation, and insertion, and methods for such manipulations are generally known in the art. For example, amino acid sequence variants of proteins can be prepared by mutations to DNA. It can also be accomplished by other forms of mutagenesis and/or by directed evolution, for example, using known mutagenesis, recombination and/or shuffling methods, combined with related screening methods, to perform single or multiple amino acid substitutions, deletions and/or insertions.

Those skilled in the art will understand that these minor amino acid changes in the HIR1 protein of the present invention can occur (e.g., naturally occurring mutations) or be generated (e.g., using r-DNA technology) without loss of protein function or activity. If these mutations occur in the catalytic domain, active site, or other functional domains of the protein, the properties of the polypeptide may be changed, but the polypeptide may retain its activity. If the mutations are not close to the catalytic domain, active site, or other functional domains, a lesser effect can be expected.

Those skilled in the art can identify the essential amino acids of the HIR1 protein according to methods known in the art, such as site-directed mutagenesis, protein evolution, or analysis of bioinformatics systems. The catalytic domain, active site, or other functional domains of the protein can also be determined by physical analysis of the structure, such as by the following techniques: nuclear magnetic resonance, crystallography, electron diffraction, or photoaffinity labeling, combined with mutations of putative key site amino acids.

TABLE 1

| Initial | Representative | Preferred |
|---|---|---|
| residue | replacement | replacement |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

The terms “polynucleotide”, “nucleotide sequence”, “nucleic acid sequence”, “nucleic acid molecule”, and “nucleic acid” are used interchangeably and include DNA, RNA, or hybrids thereof, which may be double-stranded or single-stranded.

As used herein, the term “operably linked” is intended to mean that the nucleotide sequence of interest is linked to the one or more regulatory elements in a manner that allows expression of the nucleotide sequence (e.g., in an in vitro transcription/translation system or in a host cell when the vector is introduced into the host cell).

The term “regulatory element” as used herein, is intended to include promoters, terminator sequences, leader sequences, polyadenylation sequences, signal peptide coding regions, marker genes, enhancers, internal ribosome entry sites (IRES), and other expression control elements (e.g., transcription termination signals, such as polyadenylation signals and poly-U sequences), which are described in detail in Goeddel, “GENE EXPRESSION TECHNOLOGY: METHODS IN ENZYMOLOGY” 185, Academic Press, San Diego, California (1990). In some cases, regulatory elements include those sequences that direct the constitutive expression of a nucleotide sequence in many types of host cells and those sequences that direct the nucleotide sequence to be expressed only in certain host cells (e.g., tissue-specific regulatory sequences). Tissue-specific promoters can primarily direct expression in the desired tissue of interest, such as muscle, neuron, bone, skin, blood, a specific organ (e.g., liver, pancreas), or a specific cell type (e.g., lymphocyte). In some cases, regulatory elements can also direct expression in a timing-dependent manner (e.g., in a cell cycle-dependent or developmental stage-dependent manner), which may or may not be tissue- or cell type-specific. In some cases, the term “regulatory element” encompasses enhancer elements such as WPRE; CMV enhancer; R-U5′ fragment in the LTR of HTLV-I ((Mol. Cell. Biol., Vol. 8 (1), pp. 466-472, 1988); SV40 enhancer; and intron sequences between exons 2 and 3 of rabbit β-globin (Proc. Natl. Acad. Sci. USA., Vol. 78 (3), pp. 1527-31, 1981).

As used herein, the term “promoter” has a meaning well known to those skilled in the art, which refers to a non-coding nucleotide sequence located upstream of a gene that can initiate the expression of a downstream gene. A constitutive promoter is a nucleotide sequence that, when the constitutive promoter operably linked to a polynucleotide encoding or limiting a gene product, results in the production of a gene product in a cell under most or all physiological conditions of the cell. An inducible promoter is a nucleotide sequence that, when operably linked to a polynucleotide encoding or limiting a gene product, results in the production of the gene product in the cell basically only when an inducer corresponding to the promoter is present in the cell. A tissue-specific promoter is a nucleotide sequence that, when operably linked to a polynucleotide encoding or limiting a gene product, results in the production of a gene product in the cell basically only when the cell is a cell of the tissue type corresponding to the promoter.

“Nuclear localization signal” or “nuclear localization sequence” (NLS) is an amino acid sequence that “tags” a protein to be introduced into the nucleus by nuclear transport, i.e., a protein with an NLS is transported to the nucleus. Typically, NLSs contain positively charged Lys or Arg residues exposed on the surface of the protein. Exemplary nuclear localization sequences include, but are not limited to, NLSs from: SV40 large T antigen, EGL-13, c-Myc, and TUS proteins.

The term “vector” is an element that allows the vector to be integrated into the host cell genome or to replicate autonomously in the cell independently of the genome. The vector may contain any element that ensures self-replication. It usually carries genes that are not part of the central metabolism of the cell and is usually in the form of double-stranded DNA. The choice of vector usually depends on the compatibility of the vector with the host cell into which the vector is to be introduced. If a vector is used, the choice of vector depends on the method for transforming the host cell that is well known to those skilled in the art. For example, a plasmid vector can be used.

The vector can be of the type of, for example, plasmid, virus, cosmid, phage, etc., which are well known to those skilled in the art and are described in the art. Preferably, the expression vector in the present invention is a plasmid. The expression vector may contain a promoter, a ribosome binding site for translation initiation, a polyadenylation site, a transcription terminator, an enhancer, and the like. The expression vector may also contain one or more selectable marker genes for selecting host cells containing the vector. Such selectable markers include genes encoding dihydrofolate reductase, genes that confer neomycin resistance, genes that confer resistance to tetracycline or ampicillin, etc.

The vector of the present invention may contain elements that allow the vector to be integrated into the host cell genome or to replicate autonomously in the cell independently of the genome. For integration into the host cell genome, the vector may rely on a polynucleotide sequence encoding a polypeptide or any other element of the vector suitable for integration into the genome by homologous or non-homologous recombination. Alternatively, the vector may contain additional nucleotide sequences for guiding integration into the host cell genome at an accurate location by homologous recombination on the chromosome. In order to increase the probability of integration at an accurate location, the integration element should preferably contain a sufficient number of nucleic acids, such as 100 to 10,000 base pairs, preferably 400 to 10,000 base pairs, and more preferably 800 to 10,000 base pairs, which have a high degree of identity with the corresponding target sequence to increase the probability of homologous recombination. The integration element may be any sequence homologous to the target sequence in the host cell genome. In addition, the integration element may be a non-coding or coding nucleotide sequence. On the other hand, the vector may be integrated into the genome of the host cell by non-homologous recombination. For autonomous replication, the vector may further include an origin of replication that enables the vector to replicate autonomously in the host cell. The origin of replication may be any plasmid replicon that mediates autonomous replication and functions in the cell. The term “origin of replication” or “plasmid replicon” is defined herein as a nucleotide sequence that enables a plasmid or vector to replicate in vivo.

More than one copy of the polynucleotide of the present invention may be inserted into the host cell to increase the yield of the gene product. The increase in the number of copies of the polynucleotide may be achieved by integrating at least one additional copy of the sequence into the host cell genome or by including an amplifiable selectable marker gene with the polynucleotide, and in the latter case, cells containing amplified copies of the selectable marker gene and the resulting additional copies of the polynucleotide may be selected by artificially culturing the cells in the presence of an appropriate selectable preparation.

Methods well known to those skilled in the art can be used to construct vectors containing herbicide resistance polypeptide encoding DNA sequences and appropriate transcription/translation control signals. These methods include in vitro recombinant DNA technology, DNA synthesis technology, in vivo recombination technology, etc. The DNA sequence can be effectively linked to an appropriate promoter in the vector to guide mRNA synthesis. The vector also includes a ribosome binding site for translation initiation and a transcription terminator.

Vectors suitable for use in the present invention include plasmids available from commercial channels, such as but not limited to: pBR322 (ATCC37017), pKK223-3 (Pharmacia Fine Chemicals, Uppsala, Sweden), GEMI (Promega Biotec, Madison, WI, USA), pQE70, pQE60, pQE-9 (Qiagen), pD10, psiX174 pBluescript II KS, pNH8A, pNH16a, pNH18A, pNH46A (Stratagene), ptrc99a, pKK223-3, pKK233-3, pDR540, pRIT5 (Pharmacia), pKK232-8, pCM7, pSV2CAT, pOG44, pXT1, pSG (Stratagene), pSVK3, pBPV, pMSG, and pSVL (Pharmacia), etc.

The term “plant” is to be understood as any differentiated multicellular organism capable of photosynthesis, including crop plants at any stage of maturity or development, in particular monocots or dicots, vegetable crops, including artichoke, Brussels sprouts, arugula, leek, asparagus, lettuce (e.g., head lettuce, leaf lettuce, long leaf lettuce), bok choy, malanga, melons (e.g., melon, watermelon, crenshaw, honeydew, roman melon), rape crops (e.g., Brussels sprouts, cabbage, cauliflower, broccoli, curly kale, kale, Chinese cabbage, bok choy), cardoon, carrot, napa, okra, onion, celery, parsley, chickpea, European parsnip, chicory, pepper, potato, cucurbit (e.g., zucchini, cucumber, zucchini squash, cushaw, pumpkin), radish, dry bulb onion, rutabaga, purple eggplant (also called eggplant), Solomon's ginseng, Sonchus brachyotus, scallion, Cichorium endivia, garlic, spinach, green onions, cushaw, greens, beet (sugar beet and mangel), sweet potato, Tangyin lettuce, wasabi, tomato, turnip, and spice; fruits and/or vine crops, such as apple, apricot, cherry, nectarine, peach, pear, plum, prune, cherry, quince, almond, chestnut, hazelnut, pecan, pistachio, walnut, citrus, blueberry, boysenberry, cranberry, ribe nigrum, loganberry, raspberry, strawberry, blackberry, grape, avocado, banana, kiwi, persimmon, pomegranate, pineapple, tropical fruit, pome, melon, mango, papaya, and lychee; field crops such as clover, alfalfa, evening primrose, meadowfoam, corn/maize (fodder corn, sweet corn, popcorn), hops, jojoba, peanut, rice, safflower, small grain cereal crops (barley, oat, rye, wheat, etc.), sorghum, tobacco, kapok, legumes (beans, lentil, pea, soybean), oil plants (oilseed rape, mustard, poppy, olive, sunflower, coconut, castor oil plants, cocoa bean, peanut), Arabidopsis, fiber plants (cotton, flax, hemp, jute), Lauraceae (cinnamon, camphenone), or a plant such as coffee, sugar cane, tea, and natural rubber plants; and/or bedding plants, such as flowering plants, cactus, succulents and/or ornamentals, and trees, such as forests (broadleaf trees and evergreen trees, such as coniferous tree), fruit trees, ornamental trees, and nut-bearing trees, as well as shrubs and other seedlings.

The term “unwanted plants” is understood to mean plants that affect the normal growth of desired plants (such as crops) and have no practical or application value, and can include weeds, such as dicot weeds and monocot weeds. Dicot weeds include, but are not limited to, weeds from the genera Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, and Taraxacum. Monocot weeds include, but are not limited to, weeds from the genera Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, and Apera. The unwanted plants may also include other plants different from the desired cultivated plant, for example, crops such as soybeans that grow naturally in part or in small amounts in an area where rice is cultivated.

In the present invention, the term “plant tissue” or “plant part” includes plant cells, protoplasts, plant tissue culture, plant callus, plant pieces, and plant embryos, pollen, ovules, seeds, leaves, stems, flowers, branches, seedlings, fruits, cores, spikes, roots, root tips, anthers, etc.

In the present invention, “plant cells” should be understood as any cells from or found in plants, which can form, for example: undifferentiated tissues such as callus, differentiated tissues such as embryos, plant components, plants, or seeds.

In the present invention, the term “gene editing” technology includes CRISPR technology, transcription activator-like effector nuclease (TALEN) technology, and Zinc-finger nuclease (ZFN) technology. The gene editing tools referred to in CRISPR technology include guide RNA, Cas proteins (such as Cas9, Cpf1, Cas12b, etc.). The gene editing tools referred to in TALEN technology are restriction enzymes that can cut specific DNA sequences, which include a TAL effector DNA binding domain and a DNA cleavage domain. The gene editing tools referred to in ZFN technology are also restriction enzymes that can cut specific DNA sequences, which include a zinc finger DNA binding domain and a DNA cleavage domain. It is well known to those skilled in the art that by constructing nucleotides encoding gene editing tools and other regulatory elements into a suitable vector and then transforming cells, the editing of the genome in the cell can be achieved, and the types of editing include gene knockout, insertion, and base editing.

In the present invention, the term “cultivation” includes the site for cultivating the plant of the present invention, such as soil, and also includes, for example, plant seeds, plant seedlings, and grown plants. The term “control of unwanted plants” refers to an amount of herbicide sufficient to affect the growth or development of unwanted plants, such as weeds, by preventing or inhibiting the growth or development of unwanted plants, or by killing the unwanted plants. Advantageously, the effective amount for controlling unwanted plants does not significantly affect the growth and/or development of the plant seeds, plant seedlings, or plants of the present invention. Those skilled in the art can determine such an effective amount for controlling unwanted plants by routine experiments.

### Main Advantages of the Present Invention

The present invention provides an application of an HIR1 protein in the preparation of a herbicide-resistant plant, and the plant has a strong resistance to herbicides after the HIR1 gene protein is overexpressed.

### DESCRIPTION OF THE DRAWINGS

FIG. 1. Anc689BE4max-nCas9 base editing vector.

FIG. 2. Resistance of rice YX6 mutant to Isoxaflutole, where NIP is wild-type Nipponbare rice.

FIG. 3. Analysis of differentially expressed genes in YX6-R plants relative to YX6-S plants, with arrows indicating the herbicide-resistant gene LOC_Os02g09720.

FIG. 4. Structure of the LOC_Os02g09720 gene and its promoter region in YX6-R resistant plants.

FIG. 5. Genotype analysis of the LOC_Os02g09720 promoter region in YX6-R resistant plants, YX6-S sensitive plants, and YX6-R/S heterozygous plants.

FIG. 6. Resistance of wild-type Nipponbare plant NIP, resistant plant YX6, wild-type Nipponbare HIR1 knockout plant hir1 (NIP), and resistant plant YX6 HIR1 knockout plant hir1 (YX6) to Isoxaflutole.

FIG. 7. Resistance of wild-type Nipponbare plant WT (HIR1), rice YX6 mutant (OE-HIR1), and wild-type Nipponbare HIR1 knockout plant (hir1) to Isoxaflutole. Where, Mock is a group not treated with Isoxaflutole.

FIG. 8. Resistance of wild-type Nipponbare plants WT and rice YX6 mutant (OE-HIR1) under the action of 400 μM concentration of Mesotrione, 400 μM concentration of Tembotrione, 24 μM concentration of quinotrione-methyl, 400 μM concentration of Topramezone, and 400 μM concentration of Pyrasulfotole. Where, Mock is a group not treated with herbicides.

FIG. 9. Phylogenetic analysis of OsHIR1 and its related proteins.

FIG. 10. Herbicide resistance of ZmHIR1 transgenic plants.

FIG. 11. Herbicide resistance of SqHIR1 transgenic plants.

### SPECIFIC IMPLEMENTATIONS

The present invention is further described below in conjunction with the examples. The following description is only for preferred embodiments of the present invention and does not limit the present invention to other forms. Any technician familiar with the profession may use the above disclosed technical content to change it into an equivalent embodiment of the same change. Any simple modification or equivalent change made to the following embodiments based on the technical essence of the present invention without departing from the content of the scheme of the present invention falls within the protection scope of the present invention.

The following experimental content is combined with the examples to further explain the present invention. All methods and operations described in these embodiments are provided by way of example and should not be construed as limiting. For methods of DNA operations, please refer to Current Protocols in Molecular Biology, Volumes 1 and 2, Ausubel F. M. Greene Publishing Associates and Wiley Interscience, 1989, Molecular Cloning, T. Maniatis et al., 1982, or Sambrook J. and Russell D., 2001, Molecular Cloning: a laboratory manual, version 3.

### Example 1. Discovery of Rice HPPD Herbicide Resistance Mutants

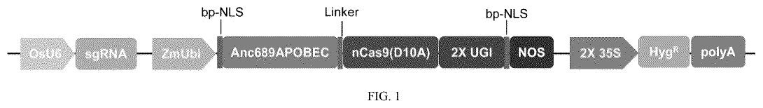

The cytosine base editor (CBE) can achieve C/G->T/A base conversion within a certain sequence window, and the Anc689BE4max-nCas9 base editor (as shown in FIG. 1) is optimized based on the first generation of CBE. The results of its application in rice show that it can greatly improve the efficiency of base conversion. In order to explore genes or mutations that are resistant to HPPD herbicides, the inventors used the base editor Anc689BE4max-nCas9 as a vector and designed targeted sgRNA to screen the mutation of the target enzymes and related genes of rice HPPD herbicides.

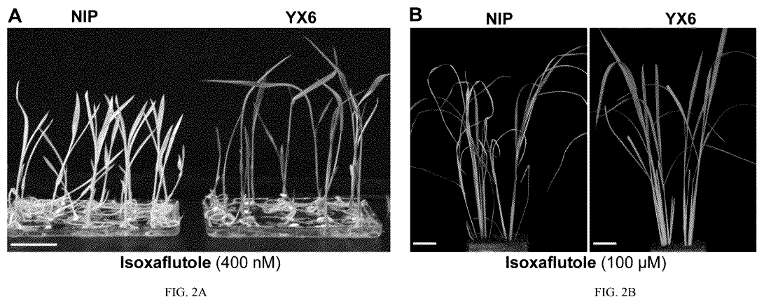

In this study, the base editor mediates genetic transformation through Agrobacterium, and the recipient variety was Nipponbare (NIP). After obtaining TO transgenic plants, the genotype of the target site was identified by PCR and sequencing, and the mutant individuals were transplanted to the field and T1 generation seeds were harvested. In order to verify the resistance of the mutant to herbicides, the harvested T1 generation seeds were dehulled, disinfected, and then inoculated on ½ MS medium with different HPPD herbicides. We selected the herbicide isoxaflutole, and the final concentration was set to 400 nM, while the tolerance concentration of wild-type Nipponbare rice to isoxaflutole during the seed germination stage was about 100 nM. Seed germination and seedling growth status were observed and counted 10 days after inoculation. We found that most seeds of the transgenic plant numbered YX6 in the T1 generation could germinate normally on the medium added with isoxaflutole, and the seedlings remained green normally, while the wild-type Nipponbare rice showed obvious bleaching symptoms of phytotoxicity (as shown in FIG. 2A). To further confirm its resistance, we transplanted the green seedlings of YX6 into the greenhouse and sprayed them with 100 μM isoxaflutole one month later. The tolerance concentration of wild-type Nipponbare rice to isoxaflutole during the seedling stage was about 40 μM. After two weeks, the phenotype was observed, and it was found that the wild-type Nipponbare rice was completely bleached and withered, while the plants of YX6 were not affected at all (as shown in FIG. 2B), indicating that the YX6 mutant has a strong resistance to isoxaflutole.

### Example 2 Location of Candidate Genes for Herbicide Resistance in Rice YX6 Mutant

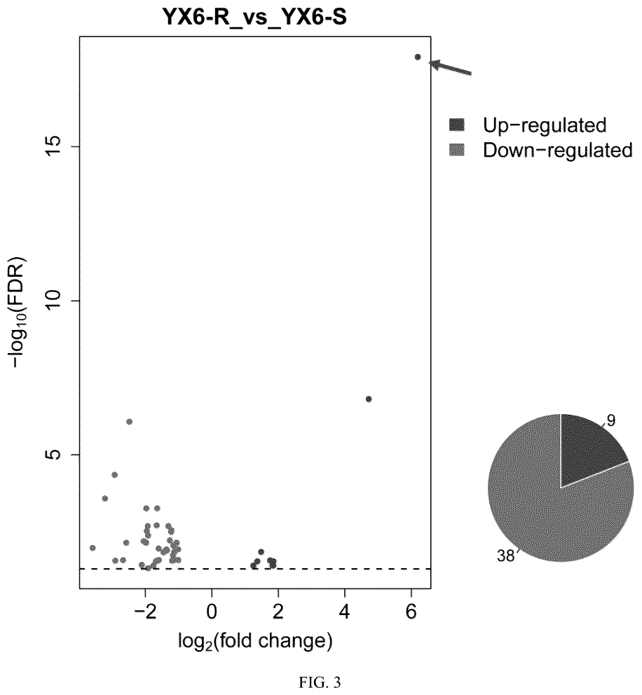

By identifying herbicide resistance in more T1 generation seeds of the YX6 mutant, we found that its resistance ratio conformed to the segregation ratio of 3:1, indicating that the gene controlling its herbicide resistance is a dominant single gene. In order to locate the gene, we propagated the phenotypically identified T1 generation seeds for two consecutive generations, namely T2 and T3 generations, and herbicide phenotypes were identified in each generation. In the T3 generation, stable homozygous-resistant plants (YX6-R), homozygous-sensitive plants (YX6-S), and heterozygous plants (YX6-R/S) whose resistance continued to separate were obtained. We sampled homozygous-resistant plants and homozygous-sensitive plants for RNAseq experiments. Analysis of differentially expressed genes showed that the expression level of a gene in the YX6-R plant was abnormally higher than that in the YX6-S population (as shown in FIG. 3).

Further analysis showed that the relative expression of this gene in resistant plants was about 73 times that of sensitive plants. The gene was numbered LOC_Os02g09720 in the rice genome, encoding 1,245 amino acids, and annotated as a multidrug resistance protein of unknown function (Table 1). Blast analysis of the predicted amino acid sequence showed that the gene was a member of the ABC transporter family.

TABLE 1
Expression analysis and genomic annotation of the putative resistance gene

| | RPKM | | | | | | | | expressed |
|---|---|---|---|---|---|---|---|---|---|
| Gene | R-1 | R-2 | R-3 | S-1 | S-2 | S-3 | R_vs_S_logFC | R_vs_S_FDR | protein |
| LOC_Os02g09720 | 344.45 | 350.61 | 401.63 | 11.05 | 1.94 | 2.00 | 6.193 | 1.22E−18 | multidrug |
| (GenBank ID: | | | | | | | | | resistance |
| NP_001396300.1) | | | | | | | | | protein. |

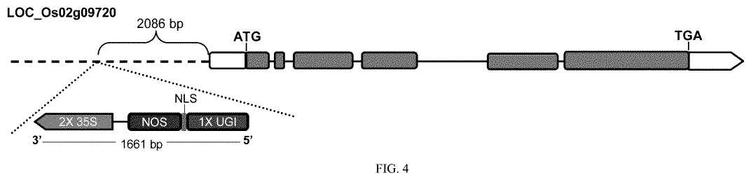

Full-length amplification and sequencing of the LOC_Os02g09720 gene showed that the coding region sequence was completely consistent in resistant plants, sensitive plants, and wild-type plants. However, in the resistant plants, we found that a reverse sequence of 1,661 bp length from the base editing vector Anc689BE4max-nCas9, including the complete 2×35S promoter element, was inserted in the promoter region of the gene, about 2 kb upstream of the ATG start codon (as shown in FIG. 4). In addition, the insertion of the exogenous fragment resulted in the loss of a 48 bp sequence at the corresponding position of the original promoter region.

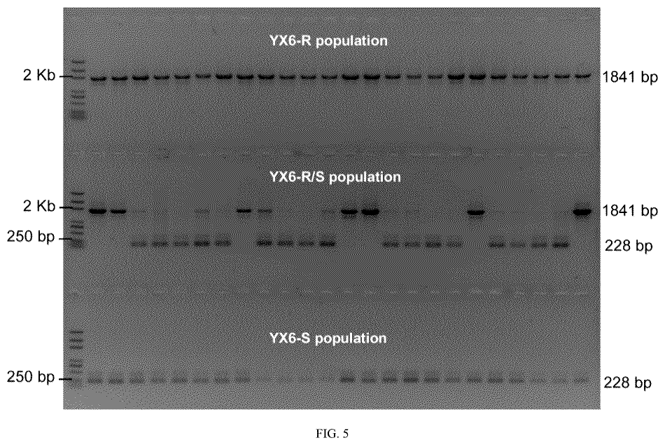

We further randomly selected 24 plants from each of the homozygous resistant population (YX6-R), the homozygous sensitive population (YX6-S), and the heterozygous population (YX6-R/S) and performed PCR amplification on the promoter region of the LOC_Os02g09720 gene. We found that all the plants in the homozygous resistant population had an insertion of an exogenous fragment of about 1,661 bp (PCR amplification product was 1,841 bp), while no such exogenous fragment was inserted in sensitive plants (PCR amplification product was 228 bp), and the genotype of the heterozygous population was between the two (as shown in FIG. 5). This result indicates that the herbicide resistance of rice plants is closely related to the LOC_Os02g09720 gene. We speculate that the 2×35S promoter reversely drives the expression of LOC_Os02g09720, resulting in a significant increase in the expression of this gene in resistant plants, thereby enhancing resistance to isoxaflutole.

### Example 3 Confirmation of Candidate Genes for Herbicide Resistance in Rice YX6 Mutants

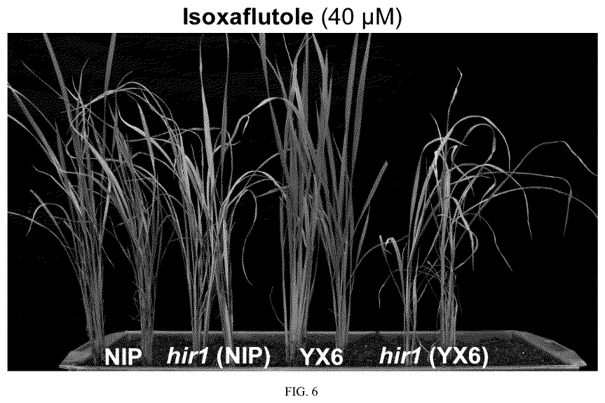

To further confirm the relationship between gene LOC_Os02g09720 and the herbicide isoxaflutole, we designed two different sgRNA targets in the coding region of gene LOC_Os02g09720 and knocked out the gene using CRISPR/Cas9 in the wild-type Nipponbare rice and YX6 resistant rice backgrounds. After obtaining TO transgenic plants, the two target regions were sequenced, and homozygous knockout plants were selected and sprayed with herbicides together with wild-type plants and YX6 resistant plants. The concentration of isoxaflutole was set to 40 μM. The phenotype was observed two weeks later. As shown in FIG. 6, the wild-type plants were bleaching and wilting, but still survived; while the YX6 plants were not affected and grew normally; the plants knocked out LOC_Os02g09720 in the wild-type and YX6 backgrounds were bleached, withered, and died gradually, showing a higher sensitivity than the wild-type plants. This result shows that LOC_Os02g09720 is a resistance gene to the herbicide isoxaflutole and is also the first endogenous isoxaflutole resistance gene discovered in plants. We named this gene HIR1 (HPPD INHIBITOR RESISTANCE 1), and its encoded protein is HIR1 protein. The amino acid sequence is shown in SEQ ID No.1. The genotype of the wild-type Nipponbare plant is HIR1, and the knockout plant is hir1. In the YX6 resistant plant, the expression level of this gene is significantly increased, and its genotype can be recorded as OE-HIR1.

### Example 4 Herbicide Resistance Strength and Resistance Type of Rice YX6 Mutant (OE-HIR1)

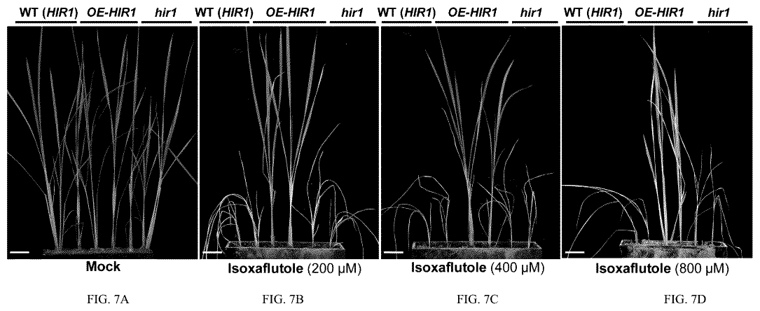

In order to further determine the resistance strength of rice YX6 mutant (OE-HIR1), we set up three high concentration gradients of isoxaflutole to spray it (200 μM, 400 μM, 800 M), with wild-type Nipponbare plants (HIR1) and wild-type Nipponbare HIR1 gene knockout plants (hir1) as controls. The results showed that the YX6 mutant (OE-HIR1) could still grow normally even under the treatment of 800 μM isoxaflutole, and the leaves did not bleach or wither, and there was no obvious phytotoxicity (as shown in FIG. 7). The tolerance range of rice YX6 mutant (OE-HIR1) to isoxaflutole may be higher, even reaching 1200-1500 μM, while the tolerance concentration of wild-type Nipponbare plants to isoxaflutole was about 40 μM, and its resistance is estimated to be increased by 30 times. The above results show that overexpression of HIR 1 in rice can significantly improve resistance to isoxaflutole.

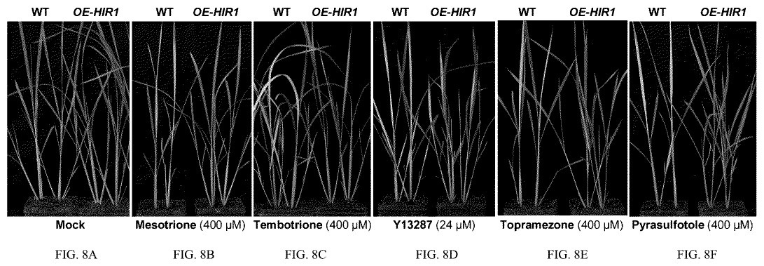

HPPD herbicides mainly include triketones, pyrazolones, isoxazolones, diketonitriles, and benzophenones. In order to determine whether the rice YX6 mutant (OE-HIR1) is resistant to other HPPD herbicides, we selected the herbicides: mesotrione, tembotrione, an unlisted quinotrione-methyl analog (Y13287), topramezone, and pyrasulfotole, and sprayed them with wild-type Nipponbare rice as the control. As shown in FIG. 8, under the action of 400 μM concentration of mesotrione, 400 μM concentration of tembotrione, 24 μM concentration of quinotrione-methyl analog, 400 μM concentration of topramezone, and 400 μM concentration of pyrasulfotole, the wild-type plants had all bleached and suffered certain phytotoxicity, but the YX6 mutant (OE-HIR1) had no phytotoxicity and could grow normally without bleaching. The resistance of the YX6 mutant (OE-HIR1) to different herbicides is: sulfone pyrazone>topramezone≈tembotrione>mesotrione≈quinotrione-methyl analog. This result shows that the endogenous resistance gene HIR1 of rice has resistance to various types of HPPD herbicides and has very important application value in crop breeding and agricultural production.

### Example 5 Herbicide Resistance of OsHIR1 Homologous Proteins

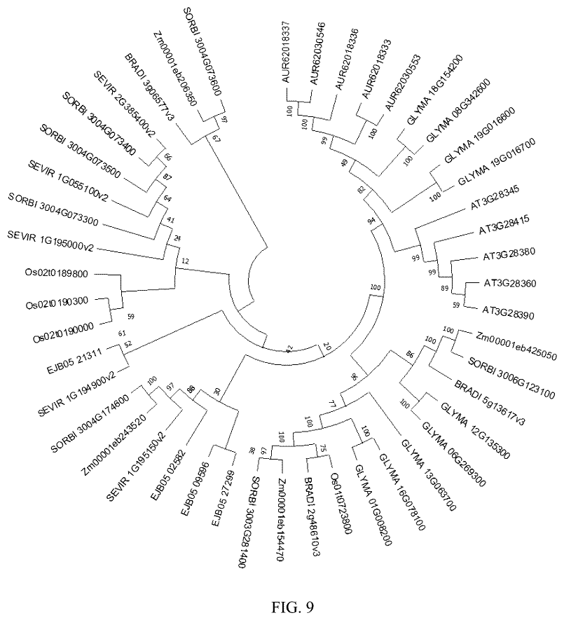

The rice OsHIR1 protein (the amino acid sequence as shown in SEQ ID No. 1) in the above examples was subjected to bioinformatics analysis, and the phylogenetic analysis of OsHIR1 and its related proteins is shown in FIG. 9. There are many OsHIR1 homologous proteins in rice, corn, sorghum, barley, wheat, soybean, quinoa, Arabidopsis, and Panicum miliaceum, as shown in the following table:

| | | Amino | |
|---|---|---|---|
| | | acid | |
| Species | Gene | length | Similarity |
| Rice | LOC_Os02g09720 (OsHIR1) | 1245 | 100% |
| | NCBI Reference Sequence: | 1243 | 92% |
| | XP_015625026.1 | | |
| | LOC_Os01g52550 | 1234 | 53% |
| Corn | Zm00001eb206350 | 1243 | 91% |
| | (NCBI Sequence ID: AQK60189.1) | | |
| | Zm00001eb243520 | 1256 | 76% |
| | Zm00001eb154470 | 1231 | 53% |
| Sorghum | OQU84543 | 1236 | 91% |
| | (NCBI Reference Sequence: | | |
| | XP_002453447.2) | | |
| | EES06419 | 1251 | 79% |
| | OQU84542 | 1262 | 78% |
| Barley | GenBank: KAE8801832.1 | 1238 | 90% |
| Wheat | TraesCS6A02G171800 | 1238 | 90% |
| | (GenBank: KAF7078743.1) | | |
| Soybean | KRH18485 | 1249 | 66% |
| | KRG99564 | 1252 | 64% |
| | KRH46566 | 1259 | 65% |
| Quinoa | AUR62030553 | 1242 | 69% |
| | AUR62018333 | 1148 | 64% |
| Arabidopsis | AT3G28345 | 1240 | 84% |
| Panicum | GenBank: RLN07167.1 | 1245 | 91% |
| miliaceum | | | |

Among them, the gene with the highest homology to OsHIR1 in corn is ZmHIR1 (Zm00001eb206350, the amino acid sequence is SEQ ID No. 4), and the gene with the highest homology to OsHIR1 in sorghum is SqHIR1 (OQU84543, the amino acid sequence is SEQ ID No. 5).

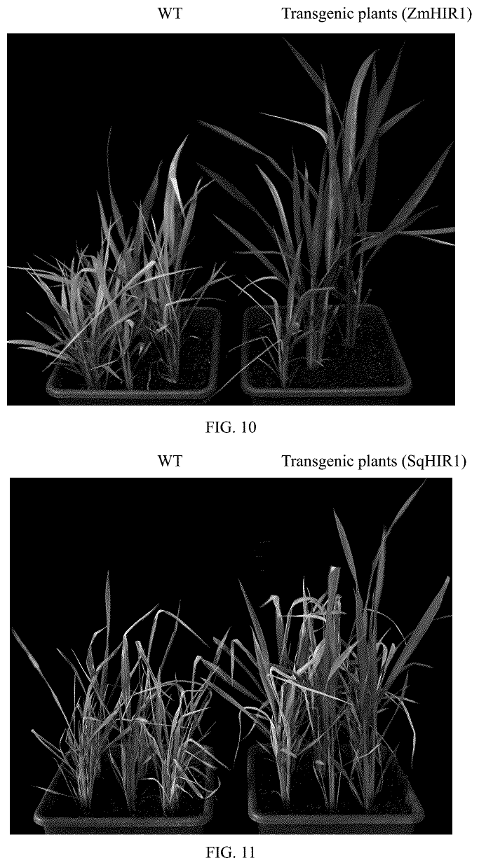

Verifying the herbicide resistance of the above two genes (ZmHIR1 and SqHIR1): cloning these two genes, respectively, and overexpressing them in the rice variety Xiushui 134. The TO generation transgenic seedlings were sprayed with 50 mg/L (139 μM) herbicide isoxaflutole for resistance identification, and the results are shown in FIGS. 10-11. According to FIG. 10, it can be seen that after spraying herbicides, the wild-type (WT) rice plants become dwarfed, yellow, and dry, while the transgenic plants (ZmHIR1) basically remain green, with normal plant height and good growth status; according to FIG. 11, it can be seen that after spraying herbicides, the wild-type (WT) rice plants were dwarfed, turned yellow and dry, while the transgenic plants (SqHIR1) basically remained green, with normal plant height and good growth status. This shows that plants overexpressing ZmHIR1 and SqHIR1 proteins are herbicide-resistant, and the HIR1 gene is a resistance gene to HPPD herbicides that is widely found in nature.

The sequence of the HIR1 protein involved in this application is as follows:

| SEQ | | | |
|---|---|---|---|
| ID | | | |
| No. | Species | GenBank | Amino acid sequence |
| 1 | Rice | NP_ | MGGGDGGAGKAKARPVFSSFMTVFMHADAADVALMVLGLLGAMGDGISTPVMLLITSRIFNDLGSG |
| | | 001396300.1 | ADIVKEFSSKVNVNARNLVFLAAASWVMAFLEGYCWARTAERQASRMRARYLRAVLRQDVEYFDL |
| | | | KKGSTAEVITSVSNDSLVVQDVLSEKVPNFVMNAAMFAGSYAVGFALLWRLTLVALPSVVLLIIPGF |
| | | | MYGRILVGLARRIREQYTRPGAIAEQAVSSARTVYSFVAERTTMAQFSAALEESARLGLKQGLAKGI |
| | | | AVGSNGITFAIWAFNVWYGSRLVMYHGYQGGTVFAVSAAIVVGGLALGSGLSNVKYFSEASSAAERI |
| | | | LEVIRRVPKIDSESDTGEELANVTGEVEFRNVEFCYPSRPESPIFVSFNLRVPAGRTVALVGGSGSG |
| | | | KSTVIALLERFYDPSAGEVMVDGVDIRRLRLKWLRAQMGLVSQEPALFATSIRENILFGKEEATAEE |
| | | | VVAAAKAANAHNFISQLPQGYDTQVGERGVQMSGGQKQRIAIARAILKSPKILLLDEATSALDTESE |
| | | | RVVQEALDLASMGRTTIVIAHRLSTIRNADIIAVMQSGEVKELGPHDELIANDNGLYSSLVRLQQTR |
| | | | DSNEIDEIGVTGSTSAVGQSSSHSMSRRFSAASRSSSARSLGDARDDDNTEKPKLPVPSFRRLLMLN |
| | | | APEWKQALMGSFSAVVFGGIQPAYAYAMGSMISVYFLTDHAEIKDKTRTYALIFVGLAVLSFLINIG |
| | | | QHYNFGAMGEYLTKRIREQMLAKILTFEIGWFDRDENSSGAICSQLAKDANVVRSLVGDRMALVIQT |
| | | | ISAVLIACTMGLVIAWRLALVMIAVQPLIIVCFYARRVLLKSMSKKSIHAQAESSKLAAEAVSNLRT |
| | | | ITAFSSQERILRLFEQSQDGPRKESIRQSWFAGLGLGTSMSLMTCTWALDFWYGGRLMAEHHISAKE |
| | | | LFQTFMILVSTGRVIADAGSMTTDLAKGADAVASVFAVLDRETEIDPDNPQGYKPEKLKGEVDIRGV |
| | | | DFAYPSRPDVIIFKGFTLSIQPGKSTALVGQSGSGKSTIIGLIERFYDPIRGSVKIDGRDIKAYNLR |
| | | | ALRRHIGLVSQEPTLFAGTIRENIVYGTETASEAEIEDAARSANAHDFISNLKDGYDTWCGERGVQL |
| | | | SGGQKQRIAIARAILKNPAILLLDEATSALDSQSEKVVQEALDRVMIGRTSVVVAHRLSTIQNCDLI |
| | | | TVLEKGTVVEKGTHASLMAKGLSGTYFSLVNLQQGGNQQVQH |
| 2 | Rice | XP_ | MGGDDRSAGKAKPVLGSFMTVFMHADAVDVVLMVLGLLGAVGDGLSMPVLLLITGSVYNNFGGGA |
| | | 015625026.1 | DNVQEFSSKVNMNARNLLFLAAGQWVMTFLEGYCWTRTAERQASRMRARYLQAVLRQDVEYFDLK |
| | | | KGSTAEVITSVANDSLVVQDVLSEKVPNFVMNAAMFVGNYAFGFALMRQLMLVALPSVVLLIIPTFM |
| | | | YGRVVVDLARRIREQYTRPGAIAEQAMSSVRTVYSFVAERTTMAQFSAALEESVRLGLKQGLAKGVA |
| | | | IGSNGITFAILAFNVWYGSRLVMSHGYKGGTVFVVSYAVIQGGLALGSVLSNVKYLSEASSAAERIL |
| | | | EVIRRVPKIDSESDTGEELGNVAGEVEFRNVKFCYPSRPESPIFVSFNLRVPAGRTVALVGGSGSGK |
| | | | STVIALLERFYDPSAGEVMVDGVDIRRLRLKWLRAQMGLVSQEPALFATSIRENILFGKEDATAEEV |
| | | | IAAAKAANAHSFISQLPQGYDTQVGERGVQMSGGQKQRIAIARAILKSPKILLLDEATSALDTESES |
| | | | VVQEALDLASMGRTTIVIAHRLSTIRNADIIAVMQSGEVKELGSHDELIANENGLYSSLVRLQQTRD |
| | | | SNEIDEIGVIGSTSALGQSSSHSMSRRFSAASRSSSVRSLGDARDADNTEKPKLPVPSFRRLLMLNA |
| | | | PEWKQALIGSFGAVVFGGIQPAFAYAMGSMISVYFLTDHAEIKDKTRTYALIFVGLAVLSFLINIGQ |
| | | | HYNFGAMGEYLTKRIREQMLAKILTFEIGWFDRDENSSGAICSQLAKDANVVRSLVGDRMALVIQTI |
| | | | SAVLIACTMGLVIAWRLALVMIAVQPLIIVCFYARRVLLKSMSKKSIHAQAESSKLAAEAVSNLRTI |
| | | | TAFSSQERILRLFDQSQDGPRKESIRQSWFAGLGLGTAMSLMACSWTIGFWYSGRLMAEHQITAKEI |
| | | | FQTFIILASTGRVIAEAGSMTTDLAKGADAVASVFAVLDRETEIDPDNPQGYKPEKLKGEVDIRRVD |
| | | | FAYPSRPDVIIFKGFTLSIQPGKSTALVGQSGSGKSTIIGLIERFYDPIRGSVKIDGRDIKAYNPRA |
| | | | LRRHIGLVSQEPTLFAGTIRENIVYGTETASEAEIEDAARSANAHDFISNLKDGYGTWCGERGVQLS |
| | | | GGQKQRIAIARAILKNPAILLLDEATSALDSQSEKVVQEALDRVMIDRTSVVVAHRLSTIQNCDLIT |
| | | | VLEKGIVVEKGTHASLMAKGPSGTYFSLVSMKQRGNQQVQQ |
| 3 | Panicum | RLN07167.1 | MGKDDGPPGTTAKKKAPPVLRSFASVFMHADAADAALMVLGLVGAMGDGLSTPVMLLITSRVFNDL |
| | miliaceum | | GSGPDLLREFSSKINENARNLVFLALANWVMAFLEGYCWARTAERQASRMRERYLRAVLRQDVEYF |
| | | | DLKVGSTSEVITSVSNDSLVVQDVLSEKVPNFVMNCSMFLGSYVVGFALLWHLTLVALPSVLLLIIP |
| | | | GFMYGRILIGLARRIRKQYTRPGAIAEQAVSSVRTVYSFVAERTTMAQFAAALEESARLGIKQGLAK |
| | | | GVAIGSNGITFTIWAFNVWYGSRLVMYHGYKGGTVFAVSAAIVVGGLALGSGLSNVKYFSEASSAAE |
| | | | RVQEVIRRVPKIDSESSAGEELPNVAGEVEFKNVEFCYPSRPETPIFVSFNLRVPAGRTVALVGGSG |
| | | | SGKSTVIALLERFYDPSAGEVTLDGVDIRRLRLKWLRAQMGLVSQEPALFATSIRENILFGKEDATE |
| | | | EEVIAVAKAANAHNFISQLPQGYHTQVGERGVQMSGGQKQRIAIARAILKSPKILLLDEATSALDTE |
| | | | SERVVQEALDLASVGRTTIVIAHRLSTIRNADMIAVMQYGEVKELGSHDELIANESGLYTALVRLQQ |
| | | | TRDSRETNEVGGTGSTSAAGQSSSHSMSRRFSAASRSSSGRSMGNAENDNNTDKPKLPVPSFRRLLM |
| | | | LNAPEWKQALMGSFSAIVFGGIQPAYAFAMGSMISIYFLTDHNEIKDKTRTYALIFVGLAVLSFLIN |
| | | | IGQHYNFGAMGEYLTKRVREQMLAKILTFEIGWFDRDENSSGAICSQLAKDANVVRSLVGDRMALVI |
| | | | QTVSAVLIACTMGLVIAWRLALVMIAVQPLIIVCFYARRVLLKSMTKKSIQAQSESSKLAAEAVSNL |
| | | | RTITAFSSQDRILRLFDQAQDGPRKESIRQSWFAGLGLGTSMSLMTCTWALDFWYGGKLMAEHHITA |
| | | | KALFQTFMILVSTGRVIADAGSMTTDLAKGADAVASVFAILDRETEIDPDNPEGYKPEKLKGEVDIR |
| | | | GVDFAYPSRPDVIIFKGFSLSIQPGKSTALVGQSGSGKSTIIGLIERFYDPLRGVVKVDGRDIKTYN |
| | | | LRALRRHIGLVSQEPTLFAGTIRENIVYGTETATEAEIENAARSANAHDFISNLKDGYDTYCGERGV |
| | | | QLSGGQKQRIAIARAILKNPAILLLDEATSALDSQSEKVVQEALDRVMVGRTSIVVAHRLSTIQNCD |
| | | | QITVLEKGIVVEKGTHASLMAKGPSGTYFGLVSLQQGGNQH |
| 4 | Corn | AQK60189.1 | MGKGGPRPAEAKKSAPALRSLASVFMHADVADVVLMVLGLVGAMGDGMSTPVMLFITSRIFNDLGS |
| | | | GPGLLQEFSSKINENARNLVFLALGNWLMAFLEGYCWARTAERQASRMRERYLRAVLRQDVEYFDL |
| | | | KVGSTSEVITSVSNDSLVVQDVLSEKVPNFVMNCSMFLGSYAVGFALLWHLTLVALPSVLLLIIPGF |
| | | | MYGRILIGLARRIREQYTRPGAIAEQAVSSVRTVYSFVAERSTMAQFSAALQESARLGVKQGLAKGV |
| | | | AIGSNGITFAIWAFNVWYGSRLVMYHGYQGGTVFAVSAAIVVGGLALGSGLSNVKYFSEASSAAERV |
| | | | QEVILRVPKIDSESSAGDELANVAGEVEFKNVEFCYPSRPETPIFVSFNLRVPAGRTVALVGGSGSG |
| | | | KSTVIALLERFYDPSAGEVTLDGVDIRRLRLKWLRAQMGLVSQEPALFATSIRENILFGKEDATGEE |
| | | | IVAAAKAANAHNFISQLPQGYDTQVGERGVQMSGGQKQRIAIARAILKSPKILLLDEATSALDTESE |
| | | | RVVQEALDLASVGRTTIVIAHRLSTIRNADMIAVMQYGEVKELGSHDDLIDNENGLYTSLVRLQQTR |
| | | | DSREANQVGGTVSTSAVGQSSSHSMSRRFSAASRSSSGRSMGDAENDNIAEKPKPPIPSFRRLLMLN |
| | | | APEWKQALMGSFSAIVFGGIQPAYAYAMGSMISIYFLADHDEIKDKTRTYALIFVALAVLSFLINIG |
| | | | QHYNFGAMGEYLTKRVREQMLAKILTFEIGWFDRDENSSGAICSQLAKDANVVRSLVGDRMALVIQT |
| | | | VSAVLIACTMGLVIAWRLALVMIAVQPLIIVCFYARRVLLKSMSKKSIQAQSESSKLAAEAVSNLRT |
| | | | ITAFSSQDRILRLFDQAQDGPRKESIRQSWFAGLGLGTSMSLMTCTWALDFWYGGKLMAERHITAKA |
| | | | LFQTFMILVSTGRVIADAGSMTTDLAKGADAVASVFAVLDRETEIDPDNPEGYKPEKLKGEVDIKGV |
| | | | DFAYPSRPDVIIFKGFSLSIQPGKSTALVGQSGSGKSTIIGLIERFYDPLRGVVKIDGKDIKTYNLR |
| | | | ALRRHIGLVSQEPTLFAGTIRENIVYGTETATEAEIENAARSANAHDFISNLKDGYDTWCGERGVQL |
| | | | SGGQKQRIAIARAILKNPAILLLDEATSALDSQSEKVVQEALDRVMVGRTSIVVAHRLSTIQNCDQI |
| | | | TVLEKGIVVEKGTHASLMAKGPSGTYFGLVSLQQGGNQH |
| 5 | Sorghum | XP_ | MGKDGPTQAAAAMAKKAPAPVMRWSFASVFMHADATDVVLMVLGLVGTMGDGFSTPVMLFITSRI |
| | | 002453447.2 | FNDLGNGPDVLQEFSSKINENARNLVFLALGCLVMAFLEGYCWARTAERQASRMRERYLRAVLRQD |
| | | | VEYFDLKVGSTSEVITSVSNDSLVVQDVLSEKLPNFVMNCAMFLGSYAVGFALLWHLTLVALPSVLL |
| | | | LIIPGFMYGRILIGLARRIREQYTRPGAIAEQAVSSVRTVYSFVAERTTMAHFSAALEESARLGIKQ |
| | | | GLAKGVAIGSNGITFAIWAFNVWYGSRLVMYHGYQGGTVFAVSAAIVVGGLALGSGLSNVKYFSEAS |
| | | | SAAERVQEVILRVPKIDSESSAGDEVANVAGDVEFKNVEFCYPSRPETPIFVSFNLRVPAGRTVALV |
| | | | GGSGSGKSTVIALLERFYDPAAGEVTLDGVDIRRLRLKWLRAQMGLVSQEPALFATSIRENILFGKE |
| | | | DATEEEVVAAAKAANAHNFISQLPQGYDTQVGERGVQMSGGQKQRIAIARAILKSPKILLLDEATSA |
| | | | LDTESERVVQEALDLASVGRTTIVVAHRLSTIRNADMIAVMQYGEVKELGSHDELIANENGLYTSLV |
| | | | RLQQTRDSREANQVGGTGSTSAAGQSSSHSMSRRFSAASRSSSGRSMGDAENDNITEKPKLPVPSFR |
| | | | RLLMLNAPEWKQALMGSFSAIVFGGIQPAYSYAMGSMISIYFLADHNEIKDKTRTYTLIFVALAVLS |
| | | | FLINIGQHYNFGAMGEYLTKRVREQMLAKILTFEIGWFDRDENSSGAICSQLAKDANVVRSLVGDRM |
| | | | ALVIQTVSAVLTACTMGLVIAWRLALVMIAVQPLIILCFYTRRVLLKSMSTKSIQAQSESSRLAAEA |
| | | | VSNLRTITAFSSQERILRLFDQAQDGPRKESIRQSWFAGLGLGTSMSLMTCTWALDFWYGGKLVAEH |
| | | | HITSKALFQTFMILVSTGRVIADAGSMTTDLAKGADAVASVFAVLDRETEIDPDNPEGYKPERLKGE |
| | | | VDIRGVDFAYPSRPDVIIFKGFSLSIQPGKSTALVGQSGSGKSTIIGLIERFYDPLRGVVKIDGKDI |
| | | | KTYNLRGLRRHIGLVSQEPTLFAGTIRENIVYGTETATEAEIENAARSANAHDFISNLKDGYDTWCG |
| | | | ERGVQLSGGQKQRIAIARAILKNPAILLLDEATSALDSQSEKVVQEALDRVMVGRTSIVVAHRLSTI |
| | | | QNCDQITVLEKGIVVEKGTHASLMAKGTSGTYFGLVSLQQGGNQH |
| 6 | Wheat | KAF | MGGAADAKKAPFGSSLVSVFMHADAADVALMVLGLVGAIGDGISTPAMLLITSRIFNDLGSGPDLLQ |
| | | 7078743.1 | EFSSKIDENARNLVFLALGCWVMAFLEGYCWSRTAERQASRMRARYLAAVLRQDVEYFDLKVGSTAE |
| | | | VIASVSNDSLVVQDVLSEKVPNFVMNAAMFFGSYAVALALLWRLTLVALPSVLLLIIPGFMYGRILI |
| | | | GLARRIREQYTRPGAVAEQAISSVRTVYSFAAERTTMAHFSAALEESTRLGIKQGLAKGIAVGSNGI |
| | | | TFAIWAFNVWYGSRLVMYHGYQGGTVFAASASIILGGLALGSGLSNVKYFSEASAAGERVLAVIRRV |
| | | | PKIDSGSDTGEELANVAGEVEFKNVEFCYPSRPESPIFASFCLRVPAGRTAALVGSSGSGKSTVVAL |
| | | | LERFYDPSGGEVALDGVDIRRLRLKWLRTQMGLVSQEPALFATSIMENILFGKEDATPEEVTAAAKA |
| | | | ANAHNFISQLPQGYDTQVGERGVQMSGGQKQRIAIARAILKSPKILLLDEATSALDTESERVVQEAL |
| | | | DLASVGRTTIVVAHRLSTIRNADMIAVMQYGEVKELGSHEELIADENGLYSSLVRLQQTRESNEVDE |
| | | | VSGAGSTSALGQSSSHSMSRRFSAASRSSSARSLGDAGDADNTEDPKLPLPSFRRLLMLNAPEWRQA |
| | | | LMGGFSAIVFGGIQPAYAYAMGSMISVYFLTDHGEIRDKTRTYALIFVALAVLSFLINIGQHYNFGA |
| | | | MGEYLTKRIREQMLTKILTFEIGWFDRDENSSGAICSQLAKDANVVRSLVGDRMALVIQTVSAVLIA |
| | | | CTMGLVIAWRLALVMIAVQPLIIVCFYARRVLLKSMSKKSIQAQSESSKLAAEAVSNLRTITAFSSQ |
| | | | DRILGLFNQAQNGPRKESIRQSWIAGLGLGTSMSLMTCTWALDFWFGGRLIAQHHITAKALFQTFMI |
| | | | LVSTGRVIADAGSMTTDLAKGADAIASVFAVLDRVTEIDPDNPEGYKPEKLKGEVDIRGVDFAYPSR |
| | | | PDVIIFKGFSLSIQSGKSTALVGQSGSGKSTIIGLIERFYDPVRGMVKIDGRDIKTYNLRALRRHIG |
| | | | LVSQEPTLFAGTIRENIVYGTETASEAEIENAARSANAHDFISNLKDGYDTWCGERGVQLSGGQKQR |
| | | | IAIARAILKNPAILLLDEATSALDSQSEKVVQEALERVMVGRTSVVVAHRLSTIQNCDLITVLDKGI |
| | | | VVEKGTHSSLMSKGPSGTYYSLVSLQQGGNQN |
| 7 | Barley | KAE | MGGAADARKSPFGSSLMSVFMHADAADVALMVLGLVGAIGDGISTPVMLLITSRIFNDLGSGPDLLQ |
| | | 8801832.1 | EFSSKIDENARNLVFLALGCWVMAFLEGYCWSRTAERQASRMRARYLAAVLRQDVEYFDLKVGSTAE |
| | | | VIASVSNDSLVVQDVLSEKVPNFVMNAAMFFGSYAVALALLWRLTVVALPSVLLLIIPGFMYGRILI |
| | | | GLARRIREQYTRPGAVAEQAISSVRTVYSFAAERATMAHFSAALEESTRLGIKQGLAKGIAVGSNGI |
| | | | TFAIWAFNVWYGSRLVMYHGYQGGTVFAASASIILGGLAQGSGLSNVKYFSEASAAGERVLAVIRRV |
| | | | PKIDSGSDTGEELANVAGEVEFKKVEFCYPSRPESPIFSSFCLRVPAGRTAALVGSSGSGKSTVVAL |
| | | | LERFYDPSGGEVALDGVDIRRLRLKWLRAQMGLVSQEPALFATSIMENILFGKEDATPEEVTAAAKA |
| | | | ANAHNFISQLPQGYDTQVGERGVQMSGGQKQRIAIARAILKSPKILLLDEATSALDTESERVVQEAL |
| | | | DLASVGRTTIVVAHRLSTIRNADMIAVMQYGEIKELGSHEELIAYENGLYSSLVRLQQTRESNEVDE |
| | | | VSGAGSTSAVGQSSSHSMSRRFSAASRSSSARSLGDAGDADNSEEPKLPLPSFRRLLMLNAPEWRQA |
| | | | LMGSLSAIVFGGIQPAYAYAMGSMISVYFLTDHDEIKDKTRAYALIFVALAVLSFLINIGQHYNFGA |
| | | | MGEYLTKRIREQMLTKILTFEIGWFDRDENSSGAICSQLAKDANVVRSLVGDRMALVIQTVSAVLIA |
| | | | CTMGLVIAWRLALVMIAVQPLIIVCFYARRVLLKSMSKKSIQAQSESSKLAAEAVSNLRTITAFSSQ |
| | | | DRILGLFNQAQNGPRKESIRQSWIAGLGLGTSMSLMTCTWALDFWFGGRLIAQHHITAKALFQTFMI |
| | | | LVSTGRVIADAGSMTTDLAKGADAIASVFAVLDRVTEIDPDNPQGYKPEKLKGEVDIRGVDFAYPSR |
| | | | PDVIIFKGFSLSIQSGKSTALVGQSGSGKSTIIGLIERFYDPVRGMVKIDGRDIKTYNLRALRQHIG |
| | | | LVSQEPTLFAGTIRENVVYGTETASEAEIENAARSANAHDFISNLKDGYDTWCGERGVQLSGGQKQR |
| | | | IAIARAILKNPAILLLDEATSALDSQSEKVVQEALERVMVGRTSVVVAHRLSTIQNCDLITVLDKG |
| | | | IVVEKGTHSSLMSKGPSGTYYSLVSLQQGGNQN |

All documents mentioned in the present invention are cited as references in this application, just as each document is cited as a reference individually. In addition, it should be understood that after reading the above teachings of the present invention, those skilled in the art can make various changes or modifications to the present invention, and these equivalent forms also fall within the scope defined by the claims attached to this application.

### Project Name: SF109-Z

### Status: Generated

### Creation Date: 2023 Apr. 10

### Sequence

### Sequence 1: “1”

| | | | Contains DNA | |
|---|---|---|---|---|
| | Molecule | | and RNA | Skipped |
| Length | Type | Organism | Segments | Sequence |
| 1245 | AA | Onyzn sadva | No | No |

### Features

| Feature Key | Feature Location | Qualifiers |
|---|---|---|
| source | 1 . . . 1245 | mol_type = protein |
| | | organism = Oryza sativa |

### Residues

| | |
|---|---|
| MGGGDGGACK AKARPVFSSF MTVFMHADAA DVALMVLGLL GAMGDGISTP VMLLITSRIF | 60 |
| NDLGSGADIV KEFSSKVNVN ARNLVFLAAA SWVMAFLEGY CWARTAERQA SRMRARYLRA | 120 |
| VLRQDVEYFD LKKGSTAEVI TSVSNDSLVV QDVLSEKYPN FVMNAAMFAG SYAVGFALLW | 180 |
| RLTLVALPSV VLLIIPGFMY GRILVGLARR IREQYTRPGA IAECAVSSAR TVYSFVAERT | 240 |
| TMAQFSAALE ESARLGLKQG LAKGIAVGSN GITFAIWAFN VWYGSRLVMY HGYQGGTVFA | 300 |
| VSAAIVVGGL ALGSGLSNVK YFSEASSAAE RILEVIRRVP KIDSESDTGE ELANVTGEVE | 360 |
| FRNVEFCYPS RPESPIFVSF NLRVPAGRTV ALVGGSGSGK STVIALLERF YDPSAGEVMV | 420 |
| DGVDIRRLRL KWLRAQMGLV SQEPALFATS IRENILFGKE EATAEEVVAA AKAANAHNFI | 480 |
| SQLPQGYDTQ VGERGVQMSG GQKQRIAIAR AILKSPKILL LDEATSALDT ESERVVQEAL | 540 |
| DLASMGRTTI VIAHRLSTIR NADIIAVMQS GEVKELGPHD ELIANDNGLY SSLVRLQQTR | 600 |
| DSNEIDEIGV TGSTSAVGQS SSHSMSRRFS AASRSSSARS LGDARDDDNT EKPKLPVPSF | 660 |
| RRLLMLNAPE WKQALMGSFS AVVFGGIQPA YAYAMGSMIS VYFLTDHAEI KDKTRTYALI | 720 |
| FVGLAVLSFL INIGQHYNFG AMGEYLTKRI REQMLAKILT FEIGWFDRDE NSSGAICSQL | 780 |
| AKDANVVRSL VGDRMALVIQ TISAVLIACT MGLVIAWRLA LVMIAVQPLI IVCFYARRVL | 840 |
| LKSMSKKSIH AQAESSKLAA EAVSNLRTIT AFSSQERILR LFEQSQDGPR KESIRQSWFA | 900 |
| GLGLGTSMSL MTCTWALDFW YGGRLMAEHH ISAKELFQTF MILVSTGRVI ADAGSMTTDL | 960 |
| AKGADAVASV FAVLDRETEI DPDNPQGYKP EKLKGEVDIR GVDFAYPSRP DVIIFKGFTL | 1020 |
| SIQPGKSTAL VGQSGSGKST IIGLIERFYD PIRGSVKIDG RDIKAYNLRA LRRHIGLVSQ | 1080 |
| EPTLFAGTIR ENIVYGTETA SEAEIEDAAR SANAHDFISN LKDGYDTWCG ERGVQLSGGQ | 1140 |
| KQRIAIARAI LKNPAILLLD EATSALDSQS EKVVQEALDR VMIGRTSVVV AHRLSTIQNC | 1200 |

Due to exceeding the specified quantity (maximum residue count of 1200 for printing), the residue sequence has been truncated.

### Sequence 2: “2”

| | | | Contains DNA | |
|---|---|---|---|---|
| | Molecule | | and RNA | Skipped |
| Length | Type | Organism | Segments | Sequence |
| 1243 | AA | Onyzn sadva | No | No |

### Features

| Feature Key | Feature Location | Qualifiers |
|---|---|---|
| source | 1 . . . 1243 | mol_type = protein |
| | | organism = Oryza sativa |

### Residues

| | |
|---|---|
| MGGDDRSAGK AKPVLGSFMT VFNHADAVDV VLMVLGLLGA VGDGLSMPVL LLITGSVYNN | 60 |
| FGGGADNVQE FSSKVNMNAR NLLFLAAGQW VMTFLEGYCW TRTAERQASR MRARYLQAVL | 120 |
| RQDVEYFDLK KGSTAEVITS VANDSLVVQD VLSEKVPNFV MNAAMFVGNY AFGFALMRQL | 180 |
| MLVALPSVVL LIIPTFMYGR VVVDLARRIR EQYTRPGAIA EQAMSSVRTV YSFVAERTTM | 240 |
| AQFSAALEES VRLGLKQGLA KGVAIGSNGI TFAILAFNVW YGSRLVMSHG YKGGTVFVVS | 300 |
| YAVIQGGLAL GSVLSNVKYL SEASSAAERI LEVIRRVPKI DSESDTGEEL GNVAGEVEFR | 360 |
| NVKFCYPSRP ESPIFVSFNL RVPAGRTVAL VGGSGSGKST VIALLERFYD PSAGEVMVDG | 420 |
| VDIRRLRLKW LRAQMGLVSQ EPALFATSIR ENILFGKEDA TAEEVIAAAK AANAHSFISQ | 480 |
| LPQGYDTOVG ERGVQMSGGQ KQRIAIARAI LKSPKILLLD EATSALDTES ESVVQEALDL | 540 |
| ASMGRTTIVI AHRLSTIRNA DIIAVMQSGE VKELGSHDEL IANENGLYSS LVRLQQTRDS | 600 |
| NEIDEIGVIG STSALGQSSS HSMSRRFSAA SRSSSVRSLG DARDADNTEK PKLPVPSFRR | 660 |
| LLMLNAPEWK QALIGSFGAV VFGGIQPAFA YAMGSMISVY FLTDHAEIKD KTRTYALIFV | 720 |
| GLAVLSFLIN IGQHYNFGAM GEYLTKRIRE QMLAKILTFE IGWFDRDENS SGAICSQLAK | 780 |
| DANVVRSLVG DRMALVIQTI SAVLIACTMG LVIAWRLALV MIAVQPLIIV CFYARRVLLK | 840 |
| SMSKKSIHAQ AESSKLAAEA VSNLRTITAF SSQERILRLF DQSQDGPRKE SIRQSWFAGL | 900 |
| GLGTAMSLMA CSWTIGFWYS GRLMAEHQIT AKEIFQTFII LASTGRVIAE AGSMTTDLAK | 960 |
| GADAVASVFA VLDRETEIDP DNPQGYKPEK LKGEVDIRRV DFAYPSRPDV IIFKGFTLSI | 1020 |
| QPGKSTALVG QSGSGKSTII GLIERFYDPI RGSVKIDGRD IKAYNPRALR RHIGLVSQEP | 1080 |
| TLFAGTIREN IVYGTETASE AEIEDAARSA NAHDFISNLK DGYGTWCGER GVQLSGGQKQ | 1140 |
| RIAIARAILK NPAILLLDEA TSALDSQSEK VVQEALDRVM IDRTSVVVAH RLSTIQNCDL | 1200 |

Due to exceeding the specified quantity (maximum residue count of 1200 for printing), the residue sequence has been truncated.

### Sequence 3: “3”

| | | | Contains DNA | |
|---|---|---|---|---|
| | Molecule | | and RNA | Skipped |
| Length | Type | Organism | Segments | Sequence |
| 1245 | AA | Panicum miliaceum | No | No |

### Features

| Feature Key | Feature Location | Qualifiers |
|---|---|---|
| source | 1 . . . 1245 | mol_type = protein |
| | | organism = Panicum miliaceum |

### Residues

| | |
|---|---|
| MGKDDGPPGT TAKKKAPPVL RSFASVFMHA DAADAALMVL GLVGAMGDGL STPVMLLITS | 60 |
| RVFNDLGSGP DLLREFSSKI NENARNLVFL ALANWVMAFL EGYCWARTAE RQASRMRERY | 120 |
| LRAVLRQDVE YFDLKVGSTS EVITSVSNDS LVVQDVLSEK VPNFVMNCSM FLGSYVVGFA | 180 |
| LLWHLTLVAL PSVLLLIIPG FMYGRILIGL ARRIRKQYTR PGAIAEQAVS SVRTVYSFVA | 240 |
| ERTTMAQFAA ALEESARLGL KQGLAKGVAI GSNGITFTIW AFNVWYGSRL VMYHGYKGGT | 300 |
| VFAVSAAIVV GGLALGSGLS NVKYFSEASS AAERVQEVIR RVPKIDSESS AGEELPNVAG | 360 |
| EVEFKNVEFC YPSRPETPIF VSFNLRVPAG RTVALVGGSG SGKSTVIALL ERFYDPSAGE | 420 |
| VTLDGVDIRR LRLKWLRAQM GLVSQEPALF ATSIRENILF GKEDATEEEV IAVAKAANAH | 480 |
| NFISQLPQGY HTQVGERGVQ MSGGQKQRIA IARAILKSPK ILLLDEATSA LDTESERVVQ | 540 |
| EALDLASVGR TTIVIAHRLS TIRNADMIAV MQYGEVKELG SHDELIANES GLYTALVRLQ | 600 |
| QTRDSRETNE VGGTGSTSAA GQSSSHSMSR RFSAASRSSS GRSMGNAEND NNTDKPKLPV | 660 |
| PSFRRLLMLN APEWKQALMG SFSAIVFGGI QPAYAFAMGS MISIYFLTDH NEIKDKTRTY | 720 |
| ALIFVGLAVL SFLINIGQHY NFGAMGEYLT KRVREQMLAK ILTFEIGWFD RDENSSGAIC | 780 |
| SQLAKDANVV RSLVGDRMAL VIQTVSAVLI ACTMGLVIAW RLALVMIAVQ PLIIVCFYAR | 840 |
| RVLLKSMTKK SIQAQSESSK LAAEAVSNLR TITAFSSQDR ILRLFDQAQD GPRKESIRQS | 900 |
| WFAGLGLGTS MSLMTCTWAL DFWYGGKLMA EHHITAKALF QTFMILVSTG RVIADAGSMT | 960 |
| TDLAKGADAV ASVFAILDRE TEIDPDNPEG YKPEKLKGEV DIRGVDFAYP SRPDVIIFKG | 1020 |
| FSLSIQPGKS TALVGQSGSG KSTIIGLIER FYDPLRGVVK VDGRDIKTYN LRALRRHIGL | 1080 |
| VSQEPTLFAG TIRENIVYGT ETATEAEIEN AARSANAHDF ISNLKDGYDT YCGERGVQLS | 1140 |
| GGQKQRIAIA RAILKNPAIL LLDEATSALD SQSEKVVQEA LDRVMVGRTS IVVAHRLSTI | 1200 |

Due to exceeding the specified quantity (maximum residue count of 1200 for printing), the residue sequence has been truncated.

### Sequence 4: “4”

| | | | Contains DNA | |
|---|---|---|---|---|
| | Molecule | | and RNA | Skipped |
| Length | Type | Organism | Segments | Sequence |
| 1243 | AA | Zea mays | No | No |

### Features

| Feature Key | Feature Location | Qualifiers |
|---|---|---|
| source | 1 . . . 1243 | mol_type = protein |
| | | organism = Zea mays |

### Residues

| | |
|---|---|
| MGKGGPRPAE AKKSAPALRS LASVFMHADV ADVVLMVLGL VGAMGDGMST PVMLFITSRI | 60 |
| FNDLGSGPGL LCEFSSKINE NARNLVFLAL GNWLMAFLEG YCWARTAERQ ASRMRERYLR | 120 |
| AVLRQDVEYF DLKVGSTSEV ITSVSNDSLV VQDVLSEKVP NFVMNCSMFL GSYAVGFALL | 180 |
| WHLTLVALPS VLLLIIPGFM YGRILIGLAR RIREQYTRPG AIAEQAVSSV RTVYSFVAER | 240 |
| STMAQFSAAL QESARLGVKQ GLAKGVAIGS NGITFAIWAF NVWYGSRLVM YHGYQGGTVF | 300 |
| AVSAAIVVGG LALGSGLSNV KYFSEASSAA ERVQEVILRV PKIDSESSAG DELANVAGEV | 360 |
| EFKNVEFCYP SRPETPIFVS FNLRVPAGRT VALVGGSGSG KSTVIALLER FYDPSAGEVT | 420 |
| LDGVDIRRLR LKWLRAQMGL VSQEPALFAT SIRENILFGK EDATGEEIVA AAKAANAHNF | 480 |
| ISQLPQGYDT QVGERGVQMS GGQKQRIAIA RAILKSPKIL LLDEATSALD TESERVVQEA | 540 |
| LDLASVGRTT IVIAHRLSTI RNADMIAVMQ YGEVKELGSH DDLIDNENGL YTSLVRLQQT | 600 |
| RDSREANQVG GTVSTSAVGQ SSSHSMSRRF SAASRSSSGR SMGDAENDNI AEKPKPPIPS | 660 |
| FRRLLMLNAP EWKQALMGSF SAIVFGGIQP AYAYAMGSMI SIYFLADHDE IKDKTRTYAL | 720 |
| IFVALAVLSF LINIGQHYNF GAMGEYLTKR VREQMLAKIL TFEIGWFDRD ENSSGAICSQ | 780 |
| LAKDANVVRS LVGDRMALVI QTVSAVLIAC TMGLVIAWRL ALVMIAVQPL IIVCFYARRV | 840 |
| LLKSMSKKSI QAQSESSKLA AEAVSNLRTI TAFSSQDRIL RLFDQAQDGP RKESIRQSWF | 900 |
| AGLGLGTSMS LMTCTWALDF WYGGKLMAER HITAKALFQT FMILVSTGRV IADAGSMTTD | 960 |
| LAKGADAVAS VFAVLDRETE IDPDNPEGYK PEKLKGEVDI KGVDFAYPSR PDVIIFKGFS | 1020 |
| LSIQPGKSTA LVGQSGSGKS TIIGLIERFY DPLRGVVKID GKDIKTYNLR ALRRHIGLVS | 1080 |
| QEPTLFAGTI RENIVYGTET ATEAEIENAA RSANAHDFIS NLKDGYDTWC GERGVQLSGG | 1140 |
| QKQRIAIARA ILKNPAILLL DEATSALDSQ SEKVVQEALD RVMVGRTSIV VAHRLSTIQN | 1200 |

Due to exceeding the specified quantity (maximum residue count of 1200 for printing), the residue sequence has been truncated.

### Sequence 5: “5”

| | | | Contains DNA | |
|---|---|---|---|---|
| | Molecule | | and RNA | Skipped |
| Length | Type | Organism | Segments | Sequence |
| 1248 | AA | Sorghum | No | No |

### Features

| Feature Key | Feature Location | Qualifiers |
|---|---|---|
| source | 1 . . . 1248 | mol_type = protein |
| | | organism = Sorghum |

### Residues

| | |
|---|---|
| MGKDGPTQAA AAMAKKAPAP VMRWSFASVF MHADATDVVL MVLGLVGTMG DGFSTPVMLF | 60 |
| ITSRIFNDLG NGPDVLQEFS SKINENARNL VFLALGCLVM AFLEGYCWAR TAERQASRMR | 120 |
| ERYLRAVLRQ DVEYFDLKVG STSEVITSVS NDSLVVQDVL SEKLPNFVMN CAMFLGSYAV | 180 |
| GFALLWHLTL VALPSVLLLL IPGFMYGRIL IGLARRIREQ YTRPGAIAEQ AVSSVRTVYS | 240 |
| FVAERTTMAH FSAALEESAR LGIKQGLAKG VAIGSNGITF AIWAFNVWYG SRLVMYHGYQ | 300 |
| GGTVFAVSAA IVVGGLALGS GLSNVKYFSE ASSAAERVQE VILRVPKIDS ESSAGDEVAN | 360 |
| VAGDVEFKNV EFCYPSRPET PIFVSFNLRV PAGRTVALVG GSGSGKSTVL ALLERFYDPA | 420 |
| AGEVTLDGVD IRRLRLKWLR AQMGLVSQEP ALFATSIREN ILFGKEDATE EEVVAAAKAA | 480 |
| NAHNFISQLP QGYDTQVGER GVQMSGGQKQ RIAIARAILK SPKILLLDEA TSALDTESER | 540 |
| VVQEALDLAS VGRTTIVVAH RLSTIRNADM IAVMQYGEVK ELGSHDELIA NENGLYTSLV | 600 |
| RLQQTRDSRE ANQVGGTGST SAAGQSSSHS MSRRFSAASR SSSGRSMGDA ENDNITEKPK | 660 |
| LPVPSFRRLL MLNAPEWKQA LMGSFSAIVF GGIQPAYSYA MGSMISIYFL ADHNEIKDKT | 720 |
| RTYTLIFVAL AVLSFLINIG QHYNFGAMGE YLTKRVREQM LAKILTFEIG WFDRDENSSG | 780 |
| AICSQLAKDA NVVRSLVGDR MALVIQTVSA VLTACTMGLV IAWRLALVMI AVQPLIILCF | 840 |
| YTRRVLLKSM STKSIQAQSE SSRLAAEAVS NLRTITAFSS QERILRLFDQ AQDGPRKESI | 900 |
| RQSWFAGLGL GTSMSLMTCT WALDFWYGGK LVAEHHITSK ALFQTFMILV STGRVIADAG | 960 |
| SMTTDLAKGA DAVASVFAVL DRETEIDPDN PEGYKPERLK GEVDIRGVDF AYPSRPDVII | 1020 |
| FKGFSLSIQP GKSTALVGQS GSGKSTIIGL IERFYDPLRG VVKIDGKDIK TYNLRGLRRH | 1080 |
| IGLVSQEPTL FAGTIRENIV YGTETATEAE IENAARSANA HDFISNLKDG YDTWCGERGV | 1140 |
| QLSGGQKQRI AIARAILKNP AILLLDEATS ALDSQSEKVV QEALDRVMVG RTSIVVAHRL | 1200 |

Due to exceeding the specified quantity (maximum residue count of 1200 for printing), the residue sequence has been truncated.

### Sequence 6: “6”

| | | | Contains DNA | |
|---|---|---|---|---|
| | Molecule | | and RNA | Skipped |
| Length | Type | Organism | Segments | Sequence |
| 1238 | AA | Triticum aestivum | No | No |

### Features

| Feature Key | Feature Location | Qualifiers |
|---|---|---|
| source | 1 . . . 1238 | mol_type = protein |
| | | organism = Triticum aestivum |

### Residues

| | |
|---|---|
| MGGAADAKKA PFGSSLVSVF MHADAADVAL MVLGLVGAIG DGISTPAMLL ITSRIFNDLG | 60 |
| SGPDLLQEFS SKIDENARNL VFLALGCWVM AFLEGYCWSR TAERQASRMR ARYLAAVLRQ | 120 |
| DVEYFDLKVG STAEVIASVS NDSLVVQDVL SEKVPNFVMN AAMFFGSYAV ALALLWRLTL | 180 |
| VALPSVLLLI IPGFMYGRIL IGLARRIREQ YTRPGAVAEQ AISSVRTVYS FAAERTTMAH | 240 |
| FSAALEESTR LGIKQGLAKG IAVGSNGITF AIWAFNVWYG SRLVMYHGYQ GGTVFAASAS | 300 |
| IILGGLALGS GLSNVKYFSE ASAAGERVLA VIRRVPKIDS GSDTGEELAN VAGEVEFKNV | 360 |
| EFCYPSRPES PIFASFCLRV PAGRTAALVG SSGSGKSTVV ALLERFYDPS GGEVALDGVD | 420 |
| IRRLRLKWLR TQMGLVSQEP ALFATSIMEN ILFGKEDATP EEVTAAAKAA NAHNFISQLP | 480 |
| QGYDTQVGER GVQMSGGQKQ RIAIARAILK SPKILLLDEA TSALDTESER VVQEALDLAS | 540 |
| VGRTTIVVAH RLSTIRNADM IAVMQYGEVK ELGSHEELIA DENGLYSSLV RLQQTRESNE | 600 |
| VDEVSGAGST SALGQSSSHS MSRRFSAASR SSSARSLGDA GDADNTEDPK LPLPSFRRLL | 660 |
| MLNAPEWRQA LMGGFSAIVF GGIQPAYAYA MGSMISVYFL TDHGEIRDKT RTYALIFVAL | 720 |
| AVLSFLINIG QHYNFGAMGE YLTKRIREQM LTKILTFEIG WFDRDENSSG AICSQLAKDA | 780 |
| NVVRSLVGDR MALVIQTVSA VLIACTMGLV IAWRLALVMI AVQPLIIVCF YARRVLLKSM | 840 |
| SKKSIQAQSE SSKLAAEAVS NLRTITAFSS QDRILGLFNQ AQNGPRKESI RQSWIAGLGL | 900 |
| GTSMSLMTCT WALDFWFGGR LIAQHHITAK ALFQTFMILV STGRVIADAG SMTTDLAKGA | 960 |
| DAIASVFAVL DRVTEIDPDN PEGYKPEKLK GEVDIRGVDF AYPSRPDVII FKGFSLSIQS | 1020 |
| GKSTALVGQS GSGKSTIIGL IERFYDPVRG MVKIDGRDIK TYNLRALRRH IGLVSQEPTL | 1080 |
| FAGTIRENIV YGTETASEAE IENAARSANA HDFISNLKDG YDTWCGERGV QLSGGQKQRI | 1140 |
| AIARAILKNP AILLLDEATS ALDSQSEKVV QEALERVMVG RTSVVVAHRL STIQNCDLIT | 1200 |

Due to exceeding the specified quantity (maximum residue count of 1200 for printing), the residue sequence has been truncated.

### Sequence 7: “7”

| | | | Contains DNA | |
|---|---|---|---|---|
| | Molecule | | and RNA | Skipped |
| Length | Type | Organism | Segments | Sequence |
| 1238 | AA | Hordeum vulgare | No | No |

### Features

| Feature Key | Feature Location | Qualifiers |
|---|---|---|
| source | 1 . . . 1238 | mol_type = protein |
| | | organism = Hordeum vulgare |

### Residues

| | |
|---|---|
| MGGAADARKS PFGSSLMSVF MHADAADVAL MVLGLVGAIG DGISTPVMLL ITSRIFNDLG | 60 |
| SGPDLLQEFS SKIDENARNL VFLALGCWVM AFLEGYCWSR TAERQASRMR ARYLAAVLRQ | 120 |
| DVEYFDLKVG STAEVIASVS NDSLVVQDVL SEKVPNFVMN AAMFFGSYAV ALALLWRLTV | 180 |
| VALPSVLLLI IPGFMYGRIL IGLARRIREQ YTRPGAVAEQ AISSVRTVYS FAAERATMAH | 240 |
| FSAALEESTR LGIKQGLAKG IAVGSNGITF AIWAFNVWYG SRLVMYHGYQ GGTVFAASAS | 300 |
| IILGGLAQGS GLSNVKYFSE ASAAGERVLA VIRRVPKIDS GSDTGEELAN VAGEVEFKKV | 360 |
| EFCYPSRPES PIFSSFCLRV PAGRTAALVG SSGSGKSTVV ALLERFYDPS GGEVALDGVD | 420 |
| IRRLRLKWLR AQMGLVSQEP ALFATSIMEN ILFGKEDATP EEVTAAAKAA NAHNFISQLP | 480 |
| QGYDTQVGER GVQMSGGQKQ RIAIARAILK SPKILLLDEA TSALDTESER VVQEALDLAS | 540 |
| VGRTTIVVAH RLSTIRNADM IAVMQYGEIK ELGSHEELIA YENGLYSSLV RLQQTRESNE | 600 |
| VDEVSGAGST SAVGQSSSHS MSRRFSAASR SSSARSLGDA GDADNSEEPK LPLPSFRRLL | 660 |
| MLNAPEWRQA LMGSLSAIVF GGIQPAYAYA MGSMISVYFL TDHDEIKDKT RAYALIFVAL | 720 |
| AVLSFLINIG QHYNFGAMGE YLTKRIREQM LTKILTFEIG WFDRDENSSG AICSQLAKDA | 780 |
| NVVRSLVGDR MALVIQTVSA VLIACTMGLV IAWRLALVMI AVQPLIIVCF YARRVLLKSM | 840 |
| SKKSIQAQSE SSKLAAEAVS NLRTITAFSS QDRILGLFNQ AQNGPRKESI RQSWIAGLGL | 900 |
| GTSMSLMTCT WALDFWFGGR LIAQHHITAK ALFQTFMILV STGRVIADAG SMTTDLAKGA | 960 |
| DAIASVFAVL DRVTEIDPDN PQGYKPEKLK GEVDIRGVDF AYPSRPDVII FKGFSLSIQS | 1020 |
| GKSTALVGQS GSGKSTIIGL IERFYDPVRG MVKIDGRDIK TYNLRALRQH IGLVSQEPTL | 1080 |
| FAGTIRENVY YGTETASEAE IENAARSANA HDFISNLKDG YDTWCGERGV QLSGGQKQRI | 1140 |
| AIARAILKNP AILLLDEATS ALDSQSEKVV QEALERVMVG RTSVVVAHRL STIQNCDLIT | 1200 |

Due to exceeding the specified quantity (maximum residue count of 1200 for printing), the residue sequence has been truncated.

What is claimed is:

1. A method for preparing a herbicide-resistant plant or conferring or enhancing a plant resistance to a herbicide, comprising using an HIR 1 (HPPD INHIBITOR RESISTANCE 1) protein, wherein the HIR1 protein comprises one of the following groups or a combination thereof:
i. an amino acid sequence of the HIR1 protein has at least 70%, at least 75%, at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity compared with SEQ ID NO: 1; and
ii. the HIR1 protein comprises the amino acid sequence shown in one of SEQ ID NOS: 1-7.

2. The method according to claim 1, wherein the HIR1 protein is overexpressed in a plant to prepare the herbicide-resistant plant or to confer or enhance the plant resistance to the herbicide.

3. The method according to claim 1, wherein the HIR1 protein is derived from a monocot or a dicot, such as comprising a rice, a corn, a sorghum, a barley, a wheat, or Panicum miliaceum.

4. The method according to claim 1, wherein the herbicide is a 4-hydroxyphenylpyruvate dioxygenase (HPPD)-inhibiting herbicide.

5. A method for preparing a herbicide-resistant plant or conferring or enhancing a plant resistance to a herbicide, comprising using a nucleic acid molecule encoding the HIR1 protein according to claim 1 or a biological material comprising the nucleic acid molecule, wherein the biological material is selected from a vector or a host cell comprising the nucleic acid molecule.

6. (canceled)

7. The method according to claim 5, wherein the HIR1 protein is overexpressed in a plant cell, a plant seed, a plant tissue, and a plant part of the plant.

8. The method according to claim 5, wherein the herbicide is an HPPD-inhibiting herbicide.

9. A method for preparing a hybrid plant, comprising hybridizing a first plant with a second plant to obtain the hybrid plant, wherein the first plant is a herbicide-resistant plant prepared by the method according to claim 5.

10. A method for controlling an unwanted plant at a plant cultivation site, comprising steps of:
(1) providing a herbicide-resistant plant prepared by the method according to claim 5; and
(2) cultivating the herbicide-resistant plant of the step (1) and applying an HPPD-inhibiting herbicide to the plant cultivation site.

11. The method according to claim 2, wherein the HIR1 protein is derived from a monocot or a dicot, comprising a rice, a corn, a sorghum, a barley, a wheat, or Panicum miliaceum.

12. The method according to claim 2, wherein the herbicide is an HPPD-inhibiting herbicide.

13. The method according to claim 5, wherein the HIR1 protein is overexpressed in a plant to prepare the herbicide-resistant plant or to confer or enhance the plant resistance to the herbicide.

14. The method according to claim 5, wherein the HIR1 protein is derived from a monocot or a dicot, comprising a rice, a corn, a sorghum, a barley, a wheat, or Panicum miliaceum.

15. The method according to claim 7, wherein the herbicide is an HPPD-inhibiting herbicide.

16. The method according to claim 9, wherein in the method for preparing the herbicide-resistant plant or conferring or enhancing the plant resistance to the herbicide, the HIR1 protein is overexpressed in a plant cell, a plant seed, a plant tissue, and a plant part of the plant.

17. The method according to claim 9, wherein in the method for preparing the herbicide-resistant plant or conferring or enhancing the plant resistance to the herbicide, the herbicide is an HPPD-inhibiting herbicide.

18. The method according to claim 10, wherein in the method for preparing the herbicide-resistant plant or conferring or enhancing the plant resistance to the herbicide, the HIR1 protein is overexpressed in a plant cell, a plant seed, a plant tissue, and a plant part of the plant.

19. The method according to claim 10, wherein in the method for preparing the herbicide-resistant plant or conferring or enhancing the plant resistance to the herbicide, the herbicide is the HPPD-inhibiting herbicide.

20. A method for controlling an unwanted plant at a plant cultivation site, comprising steps of:
(1) providing a herbicide-resistant plant prepared by the method according to claim 9; and
(2) cultivating the herbicide-resistant plant of the step (1) and applying an HPPD-inhibiting-herbicide to the plant cultivation site.


SEQUENCE LISTING (7 sequences)

SEQ ID NO: 1 - protein, 1245 aa, Oryza sativa
MGGGDGGAGKAKARPVFSSFMTVFMHADAADVALMVLGLLGAMGDGISTPVMLLITSRIFNDLGSGADIVKEFSSKVNVNARNLVFLAAASWVMAFLEGYCWARTAERQASRMRARYLRAVLRQDVEYFDLKKGSTAEVITSVSNDSLVVQDVLSEKVPNFVMNAAMFAGSYAVGFALLWRLTLVALPSVVLLIIPGFMYGRILVGLARRIREQYTRPGAIAEQAVSSARTVYSFVAERTTMAQFSAALEESARLGLKQGLAKGIAVGSNGITFAIWAFNVWYGSRLVMYHGYQGGTVFAVSAAIVVGGLALGSGLSNVKYFSEASSAAERILEVIRRVPKIDSESDTGEELANVTGEVEFRNVEFCYPSRPESPIFVSFNLRVPAGRTVALVGGSGSGKSTVIALLERFYDPSAGEVMVDGVDIRRLRLKWLRAQMGLVSQEPALFATSIRENILFGKEEATAEEVVAAAKAANAHNFISQLPQGYDTQVGERGVQMSGGQKQRIAIARAILKSPKILLLDEATSALDTESERVVQEALDLASMGRTTIVIAHRLSTIRNADIIAVMQSGEVKELGPHDELIANDNGLYSSLVRLQQTRDSNEIDEIGVTGSTSAVGQSSSHSMSRRFSAASRSSSARSLGDARDDDNTEKPKLPVPSFRRLLMLNAPEWKQALMGSFSAVVFGGIQPAYAYAMGSMISVYFLTDHAEIKDKTRTYALIFVGLAVLSFLINIGQHYNFGAMGEYLTKRIREQMLAKILTFEIGWFDRDENSSGAICSQLAKDANVVRSLVGDRMALVIQTISAVLIACTMGLVIAWRLALVMIAVQPLIIVCFYARRVLLKSMSKKSIHAQAESSKLAAEAVSNLRTITAFSSQERILRLFEQSQDGPRKESIRQSWFAGLGLGTSMSLMTCTWALDFWYGGRLMAEHHISAKELFQTFMILVSTGRVIADAGSMTTDLAKGADAVASVFAVLDRETEIDPDNPQGYKPEKLKGEVDIRGVDFAYPSRPDVIIFKGFTLSIQPGKSTALVGQSGSGKSTIIGLIERFYDPIRGSVKIDGRDIKAYNLRALRRHIGLVSQEPTLFAGTIRENIVYGTETASEAEIEDAARSANAHDFISNLKDGYDTWCGERGVQLSGGQKQRIAIARAILKNPAILLLDEATSALDSQSEKVVQEALDRVMIGRTSVVVAHRLSTIQNCDLITVLEKGTVVEKGTHASLMAKGLSGTYFSLVNLQQGGNQQVQH

SEQ ID NO: 2 - protein, 1243 aa, Oryza sativa
MGGDDRSAGKAKPVLGSFMTVFMHADAVDVVLMVLGLLGAVGDGLSMPVLLLITGSVYNNFGGGADNVQEFSSKVNMNARNLLFLAAGQWVMTFLEGYCWTRTAERQASRMRARYLQAVLRQDVEYFDLKKGSTAEVITSVANDSLVVQDVLSEKVPNFVMNAAMFVGNYAFGFALMRQLMLVALPSVVLLIIPTFMYGRVVVDLARRIREQYTRPGAIAEQAMSSVRTVYSFVAERTTMAQFSAALEESVRLGLKQGLAKGVAIGSNGITFAILAFNVWYGSRLVMSHGYKGGTVFVVSYAVIQGGLALGSVLSNVKYLSEASSAAERILEVIRRVPKIDSESDTGEELGNVAGEVEFRNVKFCYPSRPESPIFVSFNLRVPAGRTVALVGGSGSGKSTVIALLERFYDPSAGEVMVDGVDIRRLRLKWLRAQMGLVSQEPALFATSIRENILFGKEDATAEEVIAAAKAANAHSFISQLPQGYDTQVGERGVQMSGGQKQRIAIARAILKSPKILLLDEATSALDTESESVVQEALDLASMGRTTIVIAHRLSTIRNADIIAVMQSGEVKELGSHDELIANENGLYSSLVRLQQTRDSNEIDEIGVIGSTSALGQSSSHSMSRRFSAASRSSSVRSLGDARDADNTEKPKLPVPSFRRLLMLNAPEWKQALIGSFGAVVFGGIQPAFAYAMGSMISVYFLTDHAEIKDKTRTYALIFVGLAVLSFLINIGQHYNFGAMGEYLTKRIREQMLAKILTFEIGWFDRDENSSGAICSQLAKDANVVRSLVGDRMALVIQTISAVLIACTMGLVIAWRLALVMIAVQPLIIVCFYARRVLLKSMSKKSIHAQAESSKLAAEAVSNLRTITAFSSQERILRLFDQSQDGPRKESIRQSWFAGLGLGTAMSLMACSWTIGFWYSGRLMAEHQITAKEIFQTFIILASTGRVIAEAGSMTTDLAKGADAVASVFAVLDRETEIDPDNPQGYKPEKLKGEVDIRRVDFAYPSRPDVIIFKGFTLSIQPGKSTALVGQSGSGKSTIIGLIERFYDPIRGSVKIDGRDIKAYNPRALRRHIGLVSQEPTLFAGTIRENIVYGTETASEAEIEDAARSANAHDFISNLKDGYGTWCGERGVQLSGGQKQRIAIARAILKNPAILLLDEATSALDSQSEKVVQEALDRVMIDRTSVVVAHRLSTIQNCDLITVLEKGIVVEKGTHASLMAKGPSGTYFSLVSMKQRGNQQVQQ

SEQ ID NO: 3 - protein, 1245 aa, Panicum miliaceum
MGKDDGPPGTTAKKKAPPVLRSFASVFMHADAADAALMVLGLVGAMGDGLSTPVMLLITSRVFNDLGSGPDLLREFSSKINENARNLVFLALANWVMAFLEGYCWARTAERQASRMRERYLRAVLRQDVEYFDLKVGSTSEVITSVSNDSLVVQDVLSEKVPNFVMNCSMFLGSYVVGFALLWHLTLVALPSVLLLIIPGFMYGRILIGLARRIRKQYTRPGAIAEQAVSSVRTVYSFVAERTTMAQFAAALEESARLGIKQGLAKGVAIGSNGITFTIWAFNVWYGSRLVMYHGYKGGTVFAVSAAIVVGGLALGSGLSNVKYFSEASSAAERVQEVIRRVPKIDSESSAGEELPNVAGEVEFKNVEFCYPSRPETPIFVSFNLRVPAGRTVALVGGSGSGKSTVIALLERFYDPSAGEVTLDGVDIRRLRLKWLRAQMGLVSQEPALFATSIRENILFGKEDATEEEVIAVAKAANAHNFISQLPQGYHTQVGERGVQMSGGQKQRIAIARAILKSPKILLLDEATSALDTESERVVQEALDLASVGRTTIVIAHRLSTIRNADMIAVMQYGEVKELGSHDELIANESGLYTALVRLQQTRDSRETNEVGGTGSTSAAGQSSSHSMSRRFSAASRSSSGRSMGNAENDNNTDKPKLPVPSFRRLLMLNAPEWKQALMGSFSAIVFGGIQPAYAFAMGSMISIYFLTDHNEIKDKTRTYALIFVGLAVLSFLINIGQHYNFGAMGEYLTKRVREQMLAKILTFEIGWFDRDENSSGAICSQLAKDANVVRSLVGDRMALVIQTVSAVLIACTMGLVIAWRLALVMIAVQPLIIVCFYARRVLLKSMTKKSIQAQSESSKLAAEAVSNLRTITAFSSQDRILRLFDQAQDGPRKESIRQSWFAGLGLGTSMSLMTCTWALDFWYGGKLMAEHHITAKALFQTFMILVSTGRVIADAGSMTTDLAKGADAVASVFAILDRETEIDPDNPEGYKPEKLKGEVDIRGVDFAYPSRPDVIIFKGFSLSIQPGKSTALVGQSGSGKSTIIGLIERFYDPLRGVVKVDGRDIKTYNLRALRRHIGLVSQEPTLFAGTIRENIVYGTETATEAEIENAARSANAHDFISNLKDGYDTYCGERGVQLSGGQKQRIAIARAILKNPAILLLDEATSALDSQSEKVVQEALDRVMVGRTSIVVAHRLSTIQNCDQITVLEKGIVVEKGTHASLMAKGPSGTYFGLVSLQQGGNQH

SEQ ID NO: 4 - protein, 1243 aa, Zea mays
MGKGGPRPAEAKKSAPALRSLASVFMHADVADVVLMVLGLVGAMGDGMSTPVMLFITSRIFNDLGSGPGLLQEFSSKINENARNLVFLALGNWLMAFLEGYCWARTAERQASRMRERYLRAVLRQDVEYFDLKVGSTSEVITSVSNDSLVVQDVLSEKVPNFVMNCSMFLGSYAVGFALLWHLTLVALPSVLLLIIPGFMYGRILIGLARRIREQYTRPGAIAEQAVSSVRTVYSFVAERSTMAQFSAALQESARLGVKQGLAKGVAIGSNGITFAIWAFNVWYGSRLVMYHGYQGGTVFAVSAAIVVGGLALGSGLSNVKYFSEASSAAERVQEVILRVPKIDSESSAGDELANVAGEVEFKNVEFCYPSRPETPIFVSFNLRVPAGRTVALVGGSGSGKSTVIALLERFYDPSAGEVTLDGVDIRRLRLKWLRAQMGLVSQEPALFATSIRENILFGKEDATGEEIVAAAKAANAHNFISQLPQGYDTQVGERGVQMSGGQKQRIAIARAILKSPKILLLDEATSALDTESERVVQEALDLASVGRTTIVIAHRLSTIRNADMIAVMQYGEVKELGSHDDLIDNENGLYTSLVRLQQTRDSREANQVGGTVSTSAVGQSSSHSMSRRFSAASRSSSGRSMGDAENDNIAEKPKPPIPSFRRLLMLNAPEWKQALMGSFSAIVFGGIQPAYAYAMGSMISIYFLADHDEIKDKTRTYALIFVALAVLSFLINIGQHYNFGAMGEYLTKRVREQMLAKILTFEIGWFDRDENSSGAICSQLAKDANVVRSLVGDRMALVIQTVSAVLIACTMGLVIAWRLALVMIAVQPLIIVCFYARRVLLKSMSKKSIQAQSESSKLAAEAVSNLRTITAFSSQDRILRLFDQAQDGPRKESIRQSWFAGLGLGTSMSLMTCTWALDFWYGGKLMAERHITAKALFQTFMILVSTGRVIADAGSMTTDLAKGADAVASVFAVLDRETEIDPDNPEGYKPEKLKGEVDIKGVDFAYPSRPDVIIFKGFSLSIQPGKSTALVGQSGSGKSTIIGLIERFYDPLRGVVKIDGKDIKTYNLRALRRHIGLVSQEPTLFAGTIRENIVYGTETATEAEIENAARSANAHDFISNLKDGYDTWCGERGVQLSGGQKQRIAIARAILKNPAILLLDEATSALDSQSEKVVQEALDRVMVGRTSIVVAHRLSTIQNCDQITVLEKGIVVEKGTHASLMAKGPSGTYFGLVSLQQGGNQH

SEQ ID NO: 5 - protein, 1248 aa, Sorghum sp.
MGKDGPTQAAAAMAKKAPAPVMRWSFASVFMHADATDVVLMVLGLVGTMGDGFSTPVMLFITSRIFNDLGNGPDVLQEFSSKINENARNLVFLALGCLVMAFLEGYCWARTAERQASRMRERYLRAVLRQDVEYFDLKVGSTSEVITSVSNDSLVVQDVLSEKLPNFVMNCAMFLGSYAVGFALLWHLTLVALPSVLLLIIPGFMYGRILIGLARRIREQYTRPGAIAEQAVSSVRTVYSFVAERTTMAHFSAALEESARLGIKQGLAKGVAIGSNGITFAIWAFNVWYGSRLVMYHGYQGGTVFAVSAAIVVGGLALGSGLSNVKYFSEASSAAERVQEVILRVPKIDSESSAGDEVANVAGDVEFKNVEFCYPSRPETPIFVSFNLRVPAGRTVALVGGSGSGKSTVIALLERFYDPAAGEVTLDGVDIRRLRLKWLRAQMGLVSQEPALFATSIRENILFGKEDATEEEVVAAAKAANAHNFISQLPQGYDTQVGERGVQMSGGQKQRIAIARAILKSPKILLLDEATSALDTESERVVQEALDLASVGRTTIVVAHRLSTIRNADMIAVMQYGEVKELGSHDELIANENGLYTSLVRLQQTRDSREANQVGGTGSTSAAGQSSSHSMSRRFSAASRSSSGRSMGDAENDNITEKPKLPVPSFRRLLMLNAPEWKQALMGSFSAIVFGGIQPAYSYAMGSMISIYFLADHNEIKDKTRTYTLIFVALAVLSFLINIGQHYNFGAMGEYLTKRVREQMLAKILTFEIGWFDRDENSSGAICSQLAKDANVVRSLVGDRMALVIQTVSAVLTACTMGLVIAWRLALVMIAVQPLIILCFYTRRVLLKSMSTKSIQAQSESSRLAAEAVSNLRTITAFSSQERILRLFDQAQDGPRKESIRQSWFAGLGLGTSMSLMTCTWALDFWYGGKLVAEHHITSKALFQTFMILVSTGRVIADAGSMTTDLAKGADAVASVFAVLDRETEIDPDNPEGYKPERLKGEVDIRGVDFAYPSRPDVIIFKGFSLSIQPGKSTALVGQSGSGKSTIIGLIERFYDPLRGVVKIDGKDIKTYNLRGLRRHIGLVSQEPTLFAGTIRENIVYGTETATEAEIENAARSANAHDFISNLKDGYDTWCGERGVQLSGGQKQRIAIARAILKNPAILLLDEATSALDSQSEKVVQEALDRVMVGRTSIVVAHRLSTIQNCDQITVLEKGIVVEKGTHASLMAKGTSGTYFGLVSLQQGGNQH

SEQ ID NO: 6 - protein, 1238 aa, Triticum aestivum
MGGAADAKKAPFGSSLVSVFMHADAADVALMVLGLVGAIGDGISTPAMLLITSRIFNDLGSGPDLLQEFSSKIDENARNLVFLALGCWVMAFLEGYCWSRTAERQASRMRARYLAAVLRQDVEYFDLKVGSTAEVIASVSNDSLVVQDVLSEKVPNFVMNAAMFFGSYAVALALLWRLTLVALPSVLLLIIPGFMYGRILIGLARRIREQYTRPGAVAEQAISSVRTVYSFAAERTTMAHFSAALEESTRLGIKQGLAKGIAVGSNGITFAIWAFNVWYGSRLVMYHGYQGGTVFAASASIILGGLALGSGLSNVKYFSEASAAGERVLAVIRRVPKIDSGSDTGEELANVAGEVEFKNVEFCYPSRPESPIFASFCLRVPAGRTAALVGSSGSGKSTVVALLERFYDPSGGEVALDGVDIRRLRLKWLRTQMGLVSQEPALFATSIMENILFGKEDATPEEVTAAAKAANAHNFISQLPQGYDTQVGERGVQMSGGQKQRIAIARAILKSPKILLLDEATSALDTESERVVQEALDLASVGRTTIVVAHRLSTIRNADMIAVMQYGEVKELGSHEELIADENGLYSSLVRLQQTRESNEVDEVSGAGSTSALGQSSSHSMSRRFSAASRSSSARSLGDAGDADNTEDPKLPLPSFRRLLMLNAPEWRQALMGGFSAIVFGGIQPAYAYAMGSMISVYFLTDHGEIRDKTRTYALIFVALAVLSFLINIGQHYNFGAMGEYLTKRIREQMLTKILTFEIGWFDRDENSSGAICSQLAKDANVVRSLVGDRMALVIQTVSAVLIACTMGLVIAWRLALVMIAVQPLIIVCFYARRVLLKSMSKKSIQAQSESSKLAAEAVSNLRTITAFSSQDRILGLFNQAQNGPRKESIRQSWIAGLGLGTSMSLMTCTWALDFWFGGRLIAQHHITAKALFQTFMILVSTGRVIADAGSMTTDLAKGADAIASVFAVLDRVTEIDPDNPEGYKPEKLKGEVDIRGVDFAYPSRPDVIIFKGFSLSIQSGKSTALVGQSGSGKSTIIGLIERFYDPVRGMVKIDGRDIKTYNLRALRRHIGLVSQEPTLFAGTIRENIVYGTETASEAEIENAARSANAHDFISNLKDGYDTWCGERGVQLSGGQKQRIAIARAILKNPAILLLDEATSALDSQSEKVVQEALERVMVGRTSVVVAHRLSTIQNCDLITVLDKGIVVEKGTHSSLMSKGPSGTYYSLVSLQQGGNQN

SEQ ID NO: 7 - protein, 1238 aa, Hordeum vulgare
MGGAADARKSPFGSSLMSVFMHADAADVALMVLGLVGAIGDGISTPVMLLITSRIFNDLGSGPDLLQEFSSKIDENARNLVFLALGCWVMAFLEGYCWSRTAERQASRMRARYLAAVLRQDVEYFDLKVGSTAEVIASVSNDSLVVQDVLSEKVPNFVMNAAMFFGSYAVALALLWRLTVVALPSVLLLIIPGFMYGRILIGLARRIREQYTRPGAVAEQAISSVRTVYSFAAERATMAHFSAALEESTRLGIKQGLAKGIAVGSNGITFAIWAFNVWYGSRLVMYHGYQGGTVFAASASIILGGLAQGSGLSNVKYFSEASAAGERVLAVIRRVPKIDSGSDTGEELANVAGEVEFKKVEFCYPSRPESPIFSSFCLRVPAGRTAALVGSSGSGKSTVVALLERFYDPSGGEVALDGVDIRRLRLKWLRAQMGLVSQEPALFATSIMENILFGKEDATPEEVTAAAKAANAHNFISQLPQGYDTQVGERGVQMSGGQKQRIAIARAILKSPKILLLDEATSALDTESERVVQEALDLASVGRTTIVVAHRLSTIRNADMIAVMQYGEIKELGSHEELIAYENGLYSSLVRLQQTRESNEVDEVSGAGSTSAVGQSSSHSMSRRFSAASRSSSARSLGDAGDADNSEEPKLPLPSFRRLLMLNAPEWRQALMGSLSAIVFGGIQPAYAYAMGSMISVYFLTDHDEIKDKTRAYALIFVALAVLSFLINIGQHYNFGAMGEYLTKRIREQMLTKILTFEIGWFDRDENSSGAICSQLAKDANVVRSLVGDRMALVIQTVSAVLIACTMGLVIAWRLALVMIAVQPLIIVCFYARRVLLKSMSKKSIQAQSESSKLAAEAVSNLRTITAFSSQDRILGLFNQAQNGPRKESIRQSWIAGLGLGTSMSLMTCTWALDFWFGGRLIAQHHITAKALFQTFMILVSTGRVIADAGSMTTDLAKGADAIASVFAVLDRVTEIDPDNPQGYKPEKLKGEVDIRGVDFAYPSRPDVIIFKGFSLSIQSGKSTALVGQSGSGKSTIIGLIERFYDPVRGMVKIDGRDIKTYNLRALRQHIGLVSQEPTLFAGTIRENVVYGTETASEAEIENAARSANAHDFISNLKDGYDTWCGERGVQLSGGQKQRIAIARAILKNPAILLLDEATSALDSQSEKVVQEALERVMVGRTSVVVAHRLSTIQNCDLITVLDKGIVVEKGTHSSLMSKGPSGTYYSLVSLQQGGNQN